(19) United States
(12) Patent Application Publication
(10) Pub. No.: US 2025/0095824 A1
(43) Pub. Date: Mar. 20, 2025

(54) NOISE-BASED TIME COMPUTERIZED SYSTEM AND METHOD FOR OVERCOMING PLATEAUS AND BURNOUTS AND SLOWING AGING

(71) Applicant: OBERON SCIENCES ILAN LTD, Kfar Tavor (IL)

(72) Inventor: Yaron ILAN, Kfar Tavor (IL)

(21) Appl. No.: 18/888,467

(22) Filed: Sep. 18, 2024

Related U.S. Application Data
(60) Provisional application No. 63/538,899, filed on Sep. 18, 2023.

(51) Int. Cl. G16H 20/70; G06Q 10/0639; G16H 50/30

(52) U.S. Cl. CPC G16H 20/70; G06Q 10/06398; G16H 50/30

(57) ABSTRACT

Provided herein are computerized systems and methods for improving function of a subject, a group of subject, a team, a company, by introducing variability into work, for overcoming burnouts and more of the same problem, improving efficiency, preventing and/or slowing aging processes, and identifying, quantifying, and implementing at least one inherent variability pattern which is based on patterns learned from a specific subject and/or group of subjects and/or companies.

### CROSS REFERENCE TO RELATED APPLICATIONS

This application claims the benefit of priority of U.S. Provisional Patent Application No. 63/538,899, filed Sep. 18, 2023, the contents of which are incorporated herein by reference in their entirety.

### TECHNICAL FIELD

The present disclosure generally relates to the field of computerized systems and methods, including algorithms used to improve efficiency and productivity, overcome plateaus and burnouts, and slow aging processes using biological or artificial noise.

### BACKGROUND

Burnouts, lack of efficiency in workplaces, and aging processes are standard. When workers suffer from burnout, their productivity drops, and they may become less innovative and more likely to make errors. If this spreads throughout an organization, it severely impacts productivity, service quality, and the bottom. The American Psychological Association states that “workplace” burnout is an occupation-related syndrome resulting from chronic workplace stress that has not been successfully managed. Burnout can be measured and quantified using validated scientific tools. It involves ongoing emotional exhaustion, psychological distance or negativity, and feelings of inefficacy—all adding up to a state where the job-related stressors are not being effectively managed by the regular rest found in work breaks, weekends, and time off. According to leading scientific research, employees who experience true workplace burnout have a: 57% increased risk of workplace absence greater than two weeks due to illness; 180% increased risk of developing depressive disorders; 84% increased risk of Type 2 diabetes; 40% increased risk of hypertension; Additionally, workplace burnout may impair short-term memory, attention, and other cognitive processes essential for daily work activities.

The “more of the same problem”, meaning people who are continually doing the same task in their workplace or personal lives, is a contributing factor to burnouts in workplaces and personal lives and can be associated with a lack of satisfaction and even depression.

These problems are not limited to workplaces but are also part of everyone's personal schedule, which is based on routines.

Due to its multifactorial nature, aging is one of the most complex biological processes involving genetic, environmental, and social factors. Aging is a complex biological process with a multifactorial nature underlined by genetic, environmental, and social factors. Variability characterizes biological processes from the genome to cellular organelles, biochemical processes, and whole organs' function. Human life expectancy continually rises, from 67 years in 2000 to 73.4 years in 2019. Sixty years and older subjects account for 25% of the total population in several European countries. By 2050, a quarter of the world population, not including Africa, may reach that proportion. As life expectancy continues to rise, a quality of life and a healthy life expectancy is needed. Keeping the aged generation active and independent reduces the aging dependency rate. It is also associated with reducing costs for the senior population suffering from more chronic morbidity and improved quality of life.

Aging processes are associated with continuous adaptation and repeatedly doing more of the same.

Biological systems are inherently variable. Variability can be detected at the genes, proteins, and cellular function level, including metabolic heterogeneity among adjacent cells, organ function, and whole-body heterogeneity. Examples are heart rate variability, breathing variability, and gate variability. Loss or change of theses normal physiologic variabilities is associated with poor prognosis.

The plateau effect occurs for individuals following a constant regular/perpetual regimen.

There are many possible causes for burnout, lowered productivity, and lack of ability to reach maximal productivity. Most of the work tasks are based on regular and repetitious regimens. As such, they are associated with adaptation and habituation. It depends on factors associated with the genetics, physiological status of the subject, background conditions, and environmental factors. A different scheme may produce different effects of similar stimuli and tasks for every subject. Constant, repetitious, perpetual work is attempted to be overcome by the use of gradualness per se, which does not enable tailoring the efforts for said subject or company and, in most cases, is based on “one regimen for all.”

Burnout and adaptation are associated with partial or even total loss of the effect of any regimen designed to overcome it, such as adding sports or music activity. Adaptation develops to some types of regimens much more rapidly than to others. The extent of adaptation depends on the individuals' genetic, phenotypic, and other factors and the type and duration of the work. Adaptation may occur within a relatively short period, contributing to non-effectiveness.

There is, therefore, a need for an individualized platform that can assist in preventing and overcoming burnout and the aging process.

### SUMMARY

The following embodiments and aspects are described and illustrated in conjunction with systems, tools, and methods, which are meant to be exemplary and illustrative, not limiting in scope. One or more of the above-described problems have been reduced or eliminated in various embodiments, while other embodiments are directed to other advantages or improvements.

According to some embodiments, there are provided computerized systems and methods for overcoming burnout and adaptation in the workplace and personal life and preventing and slowing aging using a subject-specific or company-specific regimen-tailored algorithm.

According to some embodiments, as adaptation varies among individuals and companies and is dynamic, any subject/group of subjects/company should have a specific algorithm to overcome it, continuously changing/adapted based on the multiple variables that determine it. Accordingly, subject and/or team and/or company-tailored (collectively referred to herein in some embodiments, as “entities”), continuously developing randomization-based algorithm for improving productivity and a method for slowing aging is provided herein.

According to some embodiments, there are provided more effective methods that consider the variability between subjects and their reaction to various work tasks, to improve individual and company performance. It can also improve personal life, overcome the feeling of more of the same, and slow aging. Ordinary working and personal tasks and anti-aging maneuvers benefit from a variability of the tasks. As adaptation occurs, it prohibits an ability to improve production further and prevents reaching the maximal effect that a subject or company can achieve. Therefore, the adaptation prevents the subject and the company from reaching the maximum possible performance.

According to some embodiments, there is provided a method for overcoming burnout, adaptation, and slowing of aging processes by providing a subject/group/company continuously-developing randomization-based algorithm for improving productivity and function, which includes a subject and/or company-tailored and/or type of maneuver along with a measuring the degrees of ability to perform, the degree of variability and final performance. The algorithm uses a subject and/or group of subjects and/or company-tailored, open and closed loop, continuously or semi-continuously maneuvers to continuously improve performance and overcome adaptation and slowing aging processes.

According to some embodiments, there are provided herein methods for generating three types of task tables:
a. A table for work tasks is sorted by the concentration levels/efforts/resources required for their execution. Each type of task is also prioritized, and specify the amount of time required for its accomplishment.
b. A table for extra work activities including sports, music, meals, massage, etc.
c. A table for “do nothing periods” is part of the daily schedule where no task is allocated.

According to some embodiments, there are provided methods for four types of meters that can be utilized:
a. A personal meter, where the entities (i.e., the individual, the team and/or the company) specify their concentration level, effort ability, and energy resources they anticipate for each period during the day.
b. A performance meter that measures the efficiency and overall goal achievement
c. A variability meter that measures the degree of variability in an individual, team, or company.
d. A work environment meter is based on every team member and management input.

According to some embodiments, the algorithms utilized by the systems and methods may include one or more levels:
a. Level 1 is an open loop system that matches in a random manner tasks from the work task-table into the calendar based on priorities and time required for them, and the level of concentration and effort required. It can also change tasks between workers randomly.
b. Level 2 is an open-loop system that randomly introduces extra work tasks and “do nothing periods” into the calendar to match the period pre-defined by the worker, the team, and management.
c. Level 3 is a closed-loop system that adapts the variability in inserting the task to performance according to the performance meter. It is done using an algorithm that personalizes the variability of matching the tasks to the individual, the team, and/or the company.
d. Level 4 is a closed-loop system that quantifies and introduces measures of variability into the algorithm, e.g., a low heart rate variability period leads to a high rate of variability in the tasks. Any stimulation techniques that incorporate variability measures are included. It also considers outputs from the variabilities of the tasks themselves.

According to some embodiments, there are provided methods for improving performance, overcoming burnout, and overcoming more of the same problem in any workplace and personal life.

According to some embodiments, the described algorithm can overcome plateaus in work, in personal life, prevent and/or slow down the aging process, and improve or create new types of artwork.

According to some embodiments, there are provided methods for the algorithm to continuously learn from each subject, the team, and the whole company and adapt the algorithms based on performance.

According to some embodiments, there are provided methods for the algorithm to implement data from the working environment meters based on the overall input from the individuals, the team, and the management.

According to some embodiments, there are provided methods for personalizing the algorithm to the individual, the team, and/or the whole (entire) company.

According to some embodiments, there are provided methods for the personal achievement tool to be translated into a team and/or company achievement tool, thus enabling the most out of the time in work at all group and/or company levels.

According to some embodiments, the algorithm provides methods accounting for the variability of every individual, team, and company.

According to some embodiments, there are provided methods for measuring and quantifying variability based on all types of biosensors or any invasive and non-invasive measure of individual, group, and company variability. These include but are not limited to variability sensors, such as a watch that can track heart rate variability, breathing variability, oxygen saturation variability, or any other type of variability.

According to some embodiments, there are provided methods for adding artificial variability and using measured variability.

According to some embodiments, there are provided methods for noise and variability to play a role in overcoming burnout, dealing with more of the same problem, and improving performance and efficiency.

According to some embodiments, there are provided methods for personal meters at the level of the individual, group, and company to provide the data and monitor the performance and the variability.

According to some embodiments, methods for all measures from all types of meters are implemented into the randomization algorithm that fits the period's tasks.

According to some embodiments, there are provided methods for the algorithm to account for variability in the tasks as tasks keep changing.

According to some embodiments, methods are provided for considering all types of variabilities and implementing them into the algorithm.

According to some embodiments, there are provided methods of feedback loops based on the results.

According to some embodiments, there are provided methods for accounting for variabilities in the results themselves.

According to some embodiments, there are provided methods for a continuous and dynamic algorithm that changes based on the level of variability of the task, the results, and any variability at the level of the individual, and/or the team, and/or the company.

According to some embodiments, there are methods for introducing surprise while working on a task, which can overcome the plateau in the brain and any other organ related to the effect.

According to some embodiments, there are provided methods as the human body is designed to adapt to changes; the brain and other organs adjust, and the algorithm can overcome this tendency.

According to some embodiments, there are provided methods for implementing variability to increase motivation and improve productivity.

According to some embodiments, there are provided methods to identify intra and inter-subject, team, and/or company variability patterns, identify methods to quantify them, combine them with other patterns of variability and/or with other personalized patterns from the same subject, team or company, and from other subjects, and incorporate them into methods and algorithms which are aimed at improving the function and performances of an individual, the team and the company.

According to some embodiments, there are provided methods for personalized-based inherent variability patterns that can be learned from an individual and/or a group of individuals and/or a company and can be quantified and implemented to overcome burnout, improve workplace efficiency, and prevent and/or slow the aging process.

In some embodiments, the identified variability patterns are at the level of genes, gene networks, RNA, proteins, cell apparatus, organs, proteome, metabolome, lipidome, transcriptome, whole body, or any network patterns. They comprise both intra and inter subject-type of variabilities patterns. These patterns may be constant or change over time.

In some embodiments, there are provided herein methods and/or algorithm-based methods, which are based on a subject and/or a group of individual and/or company-specific-variability patterns and used for improvement of the efficacy of work activity, slowing of aging processes, developing new art, and any other type of activity a person is doing in his workplace or personal life.

In some embodiments, there are provided herein subject, and/or group of subjects, and/or a company-tailored, variability patterns or any type of network, continuously or semi-continuously developing, open and closed-loop method(s) and/or device(s)/system(s) implementing these variability signatures for improving the function.

In some embodiments, there are provided herein method(s) and/or device(s)/system(s) applying the method(s) for improving the efficacy of work, anti-aging drugs, anti-aging activity, or any personal activity. A subject, group and/or company-tailoring is performed by implementing variability patterns, with or without other personal patterns, and may include patterns learned from other subjects into the function of these systems while adjusting them to the pre-determined goals.

According to some embodiments, the machine learning capabilities include open and closed-loop deep learning. According to some embodiments, the machine learning capabilities are configured to be operated on a set of features by receiving values. According to some embodiments, they can quantify one or more of the personalized, group-related and/or company-related variability parameters to generate a number(s)/factor(s), with or without combining them with non-variable personalized patterns and/or combining them with patterns from other subjects, groups, and/or companies and implement them into a method or an operating method and/or an algorithm for improving the function of a system. Some of these parameters may be subject, group, and/or company specific, and some may be learned from other populations or may also be random parameters that are artificially being added.

According to some embodiments, the method may improve function in healthy or aged subjects who wish to improve function, overcome burnout in the workplace, overcome doing more of the same, and overcome aging processes.

According to some embodiments, there is provided herein a method for improving function of a subject and/or a team and/or a company (entity) in an individualized way, and/or for overcoming partial or complete loss of efficiency in a workplace, as well as slowing aging processes, by identifying, quantifying, and/or implementing at least one inherent variability pattern, which is based on patterns learned from the subject and/or from other subjects, and/or groups and/or companies and or the environment, the method comprising: identifying intra subject and/or inter subjects group and/or company variability patterns, and combining them with non-variable individualized patterns and/or networks, for generating individualized-regimen-based system operating methods or algorithms; identifying one or more methods to quantify one or more variability patterns, nonlinear networks and chaotic parameters, and combining them with variability and/or non-variability individualized group-specific and/or company-specific patterns from one or more subjects; identifying methods to incorporate one or more variability and/or non-variability patterns into regimens or operating algorithms; receiving a plurality of physiological and/or pathological inherent patterns of variability with and without additional parameters related/unrelated to a target, and/or related/unrelated to the subject, the group and/or the company; applying an open or closed-loop machine learning algorithm to a plurality of target system parameters; determining subject-specific output parameters relating to at least one target function; and utilizing the subject/team/company-specific output parameters to improve the at least one target system function by using the machine learning algorithm by applying a subject-tailored continuously or semi-continuously inherent variability patterns, thereby facilitating continual improvement of the target system; and incorporating individualized parameters based on variability-patterns and other personalized, group-related and/or company-related and any other type of non-artificial and artificial random and/or variability signatures, into irregularity generating-algorithms for improving the at least one target function in workplace, personal life and slowing of aging processes.

According to some embodiments, the method may further be utilized for improving the efficiency of calendars, all types of timetables used in work and/or in personal life activities, as the function and/or performance thereof, and for individualizing regimens and/or devices and slowing aging processes and for reaching goals determined by the subject, the team and/or the company, optionally additionally using updated at least one of the output parameters.

According to some embodiments, the machine learning algorithm considers personal data, including any individualized, group, and/or company-inherent variability patterns, wherein at least one physiological and/or pathological parameter is obtained from a sensor.

According to some embodiments, the method may include providing the subject with a device or a target system, a recommended regimen, or changes to that.

According to some embodiments, providing the recommended regimen or changes is in real-time.

According to some embodiments, the machine learning capabilities may include open and/or closed-loop deep learning capabilities and are configured to operate on a set of features by receiving values.

According to some embodiments, the irregularity-generating algorithms may include random or pseudo-random number-generating algorithms.

According to some embodiments, there is provided herein a method for improving function of a system having learning capabilities, the method comprising: obtaining an intra computerized system and/or inter computerized systems variability patterns, and combining them with non-variable patterns and/or networks, for generating system-tailored operating methods or algorithms; identifying one or more methods to quantify one or more variability patterns, nonlinear networks and chaotic parameters, and combining them with variability and/or non-variability patterns from one or more systems; identifying methods to incorporate one or more variability and/or non-variability patterns into the system-tailored operating methods or algorithms; receiving a plurality of hardware and/or software inherent patterns of variability with and without additional parameters related/unrelated to the computerized system; applying a closed-loop machine learning algorithm to a plurality of parameters characterizing one or more functions of the computerized system; determining system-tailored output parameters relating to the one or more functions; and utilizing the system-tailored output parameters to improve the one or more functions by using the machine learning algorithm by applying a system-tailored continuously or semi continuously inherent variability patterns, thereby facilitating continual improvement of the computerized system; and incorporating system-tailored parameters based on variability-patterns and other system specific signatures, into irregularity generating-algorithms for improving the one or more functions.

According to some embodiments, the computerized system comprises a mechanical and electronic device.

According to some embodiments, the computerized system comprises a smartphone, a smartwatch, a tablet, or any combination thereof.

According to some embodiments, the computerized system may include an artificial neural network.

According to some embodiments, there is provided a method for at least partially overcoming, improving, preventing and/or slowing an issue comprising: burnout in a workplace, overcoming “doing more of the same”, improving efficiency of an entity, preventing and/or slowing aging processes, and/or improving function, the method comprising identifying and/or quantifying at least one variability pattern, based on patterns learned from the entity, and implementing variability measures into timetables, calendars, and/or anti-aging treatment regime or device, wherein the identifying, quantifying and/or implementing is facilitated based on:
an identification meter configured to identify and follow the ability of the entity to perform, by tracking subjective and objective measures;
a specification meter comprising data, obtained from the entity regarding concentration level, efforts, energy level, and/or resources anticipated for each period
a performance meter configured to determine entity based on measured efficiency and overall goal achievement;
a variability meter configured to measure the degree of variability in the entity, based on physiological and pathological variability measures;
a work-environment meter configured to account for inputs from team members and/or management members of a company;
a work tasks table, sorted based on concentration levels, efforts, energy and/or resources required for execution thereof, wherein each task is prioritized and specified based on time required for its accomplishment;
a table for out of (extra) work activities, comprising sports, music, meals, and/or massages;
a table with “do nothing periods”, where no task is allocated; and
applying thereon an algorithm comprising modules comprising: an open loop module configured to match, randomly, tasks from the work task table into the timetables, calendars, and/or anti-aging treatment regime treatment or device, based on priorities and time required for their accomplishment,
an extra-work tasks open look module configured to introduce extra work tasks and randomly “do nothing period” into the calendar, to match period pre-defined by the entity
a closed-loop module configured to adapts variability used when inserting the tasks to the performance, based on the performance meter, by personalizing the variability of matching the tasks to the entity.
a variability closed-loop module configured to quantify and introduce external variability measures;
to thereby at least partially overcome one or more of said issues.

According to some embodiments, the entity includes an individual (a subject), a team (a group) and/or a company.

According to some embodiments, the algorithms may include machine learning algorithms, applied on a plurality of parameters.

According to some embodiments, the implementation of variability patterns into timetables, calendars and/or anti-aging treatment regime or device comprises utilizing random generators, pseudo or partial random generators, identifying intra- and/or inter-subject, group of subjects and/or company variability patterns and combining with non-variable individualized patterns and/or networks, for generating and implementing individualized-regimen-based patterns.

According to some embodiments, the method may include at least one inherent variability pattern, comprising variability patterns related to tasks, whole body and/or network-based variability.

According to some embodiments, the method may include identifying one or more means to quantify variability patterns, nonlinear networks, and/or chaotic parameters and combining one or more thereof with variability and/or non-variability individualized patterns from one or more entities.

According to some embodiments, the method may include identifying means to incorporate one or more variability and/or non-variability patterns into anti-aging regimens.

According to some embodiments, the method may include receiving a plurality of physiological and/or pathological inherent patterns of variability with and without additional parameters related or unrelated to the entity and applying an open-loop and/or closed-loop machine learning algorithm to a plurality of target parameters.

According to some embodiments, the method may include determining entity-specific output parameters relating to at least one target function and utilizing the output parameters to improve at least one target function based on the algorithm, by applying an entity-tailored continuously or semi-continuously inherent variability patterns, thereby facilitating continual improvement of the target function.

According to some embodiments, the method may include incorporating individualized, team-derived and/or company-derived parameters, based on variability-patterns and one or more personalized signatures, into irregularity generating algorithms, for improving the at least one target function.

According to some embodiments, the method may be utilized for improving function and/or performance and/or for individualizing thereof to thereby enhance efficacy thereof for reaching goals determined by the entity or by other entities.

According to some embodiments, the method may be utilized for prevention and/or slowing of an aging process, improving function and/or performance of the entity, and/or improving the results of using a drug, a device, and/or related anti-aging procedure and maneuver and for improving their efficacy for reaching goals determined by the entity, and/or or by other entities.

According to some embodiments, the utilized-derived data comprises individualized-inherent variability patterns or company-related variability patterns, and wherein at least one of the physiological and/or pathological parameters is obtained from a sensor.

According to some embodiments, the method may further include providing to a subject, a device, or a target functional system, a recommended regimen or changes thereto.

According to some embodiments, the method may include providing recommended regimen or changes thereto in real-time.

According to some embodiments, the algorithm may include open-loop and closed-loop deep learning capabilities, are configured to operate on a set of features by receiving values thereof.

According to some embodiments, the irregularity generating algorithms comprise random or semi-random or pseudo-random number generating algorithms.

According to some embodiments, there is provided a computerized system comprising a processor configured to implement the method disclosed herein.

According to some embodiments, there is provided herein a computer-readable storage medium comprising instructions that cause one or more processors to perform the methods disclosed herein.

According to some embodiments, there is provided a method for improving the function of a computerized system having learning capabilities, the method includes:
obtaining intra-system and/or inter-system variability patterns and combining with non-variable patterns and/or networks for generating system-tailored operating algorithms;
identifying one or more means to quantify one or more variability patterns, nonlinear networks, and chaotic parameters and combining them with variability and/or non-variability patterns from one or more systems;
identifying methods to incorporate one or more variability and/or non-variability patterns into a system-tailored operating algorithm;
receiving a plurality of hardware and/or software inherent patterns of variability with and without additional parameters related/unrelated to the system;
applying an open-loop or a closed-loop machine learning algorithm to a plurality of parameters characterizing one or more functions of the system;
determining system-tailored output parameters relating to one or more functions
utilizing the system-tailored output parameters to improve one or more functions by using the machine learning algorithm by applying a system-tailored continuously or semi-continuously inherent variability patterns, thereby facilitating continual improvement of the system; and
incorporating system-tailored parameters based on variability patterns and other system-specific signatures into irregularity-generating algorithms for improving one or more functions.

According to some embodiments, the computerized system comprises calendars, timetables, goal-based algorithms, task performance monitoring, and task performance algorithms.

According to some embodiments, the computerized system may include anti-aging related treatment, device, or procedure.

According to some embodiments, the computerized system may include artworks.

According to some embodiments, the computerized system may include an artificial neural network.

According to some embodiments, there is provided a method for improving function and performance and/or for preventing, mitigating, or overcoming partial or complete loss of effect of a regimen, due to adaptation to the regimen; and/or partial or complete loss of effect of a device-generated maneuvers or stimulations administered to or used by a subject in need thereof, or non-responsiveness to challenged-regimens, and/or maximizing effect of regimens or maneuvers, the method comprising:
a. receiving a plurality of physiological or pathological parameters of the subject and/or information from the subject, a team and/or a company, and/or a device;
b. applying an open or a closed loop machine learning algorithm on the plurality of physiological or pathological parameters;
c. determining output parameters relating to subject, team and/or company-specific challenged regimens, for facilitating a continuous improvement of the regimen or device-based maneuver or stimulation, wherein the output parameters comprise regimen or maneuver parameters, thereof,
d. utilizing a subject, team and/or company-tailored, continuously or semi-continuously randomization-based or non-randomization-based algorithm, for continually improving performance; and
e. utilizing a subject-tailored, continuously or semi-continuously developing a randomization-based or non-randomization-based algorithm capable of mixing two or more work tasks, whether relevant to the target for improving function.

According to some embodiments, the method may further include updating output parameters which includes challenged-regimens-related parameters; and/or device-generated maneuver or stimulation parameters comprising amplitude, frequency, interval, and/or duration; or any combinations thereof, comprising updating changes and alterations in each of the parameters, which are of relevance to target performance.

According to some embodiments, the method may further include determining challenged regimens and/or maneuvers or stimulation parameters.

According to some embodiments, the method may further include updating regimen parameters based on data being continuously or semi-continuously learned from user(s).

According to some embodiments, the machine learning algorithm further considers personal and/or group and/or company data selected from sources comprising subject, team and/or company performance, function-related scores, parameters relevant to performance, age, weight, tasks, gender, ethnicity, geography, pathological history and/or state, temperature, metabolic rate, brain function, health status, heart, lung muscle function, blood tests, and physiological or pathological biomarkers or parameters that can be measured, directly or indirectly associated with the physiological target or the task and/or the subject, team and/or company.

According to some embodiments, at least one of the physiological or pathological parameters is obtained from a sensor.

According to some embodiments, the subject/team/company challenged-regimen, is based on a deep machine learning closed loop-irregularity, regularity, randomization, or non-randomization.

According to some embodiments, the method may further include notifying the subject/team/company in real-time.

According to some embodiments, the method may further include challenged-regimens and/or maneuvers or stimulating-generating devices to evoke a reaction by a form of external, wearable, swallowed and/or implanted device associated with improving function.

According to some embodiments, the method may further include administering the challenged regimen to the subject/team/company.

According to some embodiments, updating the challenged-regimens and/or device-generated maneuvers or stimulation parameters includes utilizing machine-learning capabilities.

According to some embodiments, the machine learning tools comprise closed-loop deep learning.

According to some embodiments, machine learning tools are configured to be operated on a set of features by receiving values.

According to some embodiments, the method may be for improving function in healthy subjects who wish to improve performance, and/or for reaching a better target, for prevention or slowing down of aging processes, and/or for improving the effect of anti-aging drugs, maneuvers and/or techniques.

According to some embodiments, wherein challenged regimens are utilized in combination with device-generated maneuvers or stimulation parameters, or with regimens of conditions wherein enhanced functioning is required, or for improving performance, for prevention or overcoming of adaptation to chronic regimens, or for continuously overcoming partial/complete loss of an effect of these regimens, and/or for improving the beneficial effects of a regimen.

According to some embodiments, there is provided a system for preventing, mitigating and/or treating partial or complete loss of effect due to adaptation to a challenged-regimen and/or used in combination with device-generated maneuvers or stimulation parameters, administered to or used by a subject, team and/or a company in need thereof, or non-responsiveness to regimens, and continuously maximizing the beneficial effect of work regimens, and/or improving function, the system being continuous, semi-continuous, conditional or non-continuous closed loop; and comprising one or more processing units configured for:
a. receiving, a plurality of physiological or pathological parameters of the subject, team and/or company, and/or information therefrom and/or device, or other sources;
b. applying a closed-loop machine learning algorithm on the plurality of physiological or pathological parameters;
c. determining output parameters relating to subject, team. and/or company-specific challenged-regimens, and/or in combination with device-generated maneuvers/stimulation parameters, for facilitating improvement of work regimens or device-based maneuvers, wherein the output parameters comprise regimen administration parameters, maneuver/stimulation parameters, or any combination thereof;
d. using a subject/group of subjects/company-tailored continuously, semi-continuously, and non-continuous information for developing randomization-based or non-randomization-based algorithms for improving function following challenged regimens and/or any maneuver that can improve the function for continuously improving the performance related to the function of the said subject/group/company.
e. using a subject/team/company-tailored continuously or semi-continuously developing a randomization-based algorithm that mixes two or more tasks, whether relevant to the task.

According to some embodiments, the machine learning algorithm is further configured to update output parameters comprising challenged-regimen and/or administration, and precisely parameters which are relevant to the regimens which are specific for the task and/or to stimulation signals; based on initial regimens parameters and/or initial stimulation parameters and/or on continuous or semi-continuous information obtained during or following the challenged-working session, and/or a maneuver which can improve the function by overcoming adaptation to maneuvers or regimes.

According to some embodiments, the machine learning algorithm further considers subject/team/company data selected from the data comprising subject/team/company performance, task-related scores, parameters relevant to performance, and physiological or pathological biomarkers or parameters that can be measured, directly or indirectly associated with the physiological target or with function and/or health status of the subject and/or subject's chronic condition that can be measured, directly or indirectly associated with the target to be achieved continuously.

According to some embodiments, at least one of the physiological or pathological parameters is obtained from a sensor.

According to some embodiments, the subject regimen or any type of maneuver/regimen/regimens is irregular.

According to some embodiments, the system may include a processor configured to notify the subject/team/company regarding regimen and/or device-generated maneuvers/stimulation regimens-relevant parameters, including relevant and irrelevant work-related parameters for administering these regimens.

According to some embodiments, the system may include a real-time alert unit/processor configured to notify/alert the subject/team/company.

According to some embodiments, the system may further include a processor configured to use a work regimen and/or to improve function or manipulate/stimulate an organ of the subject/group to evoke a reaction by a form of external/wearable/swallowed/implanted device including devices being used for improving function.

According to some embodiments, the system may include a processor configured to administer the subject's treatment regimens or lifestyle changes.

According to some embodiments, updating the challenged regimen and/or device-generated maneuver/stimulation parameters includes utilizing machine-learning capabilities.

According to some embodiments, the machine learning capabilities include deep learning.

According to some embodiments, the machine learning capabilities are configured to be operated on a set of features by receiving values.

According to some embodiments, the system is subject/team/company-specific; challenged regimens-specific, performance-specific and/or subject/group/company-specific.

According to some embodiments, the system is for improvement of the function of any task and when a subject/team/company wishes to improve their performance and to prevent or slow aging.

According to some embodiments, in the system, the closed algorithm receives input from a subject, groups of subjects, or companies for determining a change of challenged regimen relevant to improving the target or non-target function by these regimens. Any input received from the subject or groups of subjects and assessed by the algorithm for providing an output that may improve these regimens or any device-generating maneuvers/regimen/stimulation-based regimen for a subject. It can be applied to any regimen aimed at improving the function of a tissue/organ/organs. The challenged-regimens-based algorithms continuously or semi-continuously change the parameters within a predefined range to improve performance.

According to some embodiments, the goal of the system is improving function by preventing, treating, or overcoming adaptation or partial/complete loss of an effect of these regimens or lack of maximal beneficial response to these regimens, enabling a continuous improvement in the performance.

According to some embodiments, the sensor may be configured to measure any physiological or pathological parameters that can be measured, whether directly or indirectly associated with the physiological target.

According to some embodiments, an algorithm is used to improve the long-term continuous adherence of said subject to a work regimen and/or to a device-based work, and their response to any partial/complete loss of an effect to any maneuver or method or device being used for improving function.

According to some embodiments, a computerized system is provided herein configured to implement any one or any combination of the methods disclosed herein.

According to some embodiments, a computer-readable storage medium comprises instructions that cause one or more processors to perform any combination of the methods disclosed herein.

### BRIEF DESCRIPTION OF THE DRAWINGS

Examples illustrative of embodiments are described below concerning figures attached hereto. In the figures, identical structures, elements, or parts that appear in more than one figure are generally labeled with the same numeral in all the figures in which they appear. Alternatively, elements or parts appearing in more than one figure may be labeled with different numerals in the figures in which they appear. Dimensions of components and features shown in the figures are generally chosen for convenience and clarity of presentation and are not necessarily shown in scale. The figures are listed below.

FIG. 1 schematically illustrates a functional block diagram of a system that accumulates subject/group of subjects/company-related, performance-related, device-related, and/or target-related parameters according to some embodiments and based on the use of a predetermined range for each regimen.

FIG. 2 schematically illustrates a functional block diagram of the closed loop-based algorithm for improving the regimen to prevent adaptation continuously, improve target function and/or loss of response to a regimen, and improve performance, according to some embodiments. The closed-loop provides a learning method for generating a new regimen and/or maneuver/stimulation or any device that can improve the overall target and performance to be delivered to said subject.

FIG. 3 schematically illustrates a method for providing an updated regimen using said program, procedure, maneuver, or device which can improve function, and/or a combination of maneuvers, procedures, drugs, devices, and/or medical devices, and/or stimulation pattern using device or procedure or method or software which can improve function, according to some embodiments. It schematically illustrates a functional block diagram of the subject-tailored continuously or semi-continuously learning-closed loop system.

FIG. 4 schematically illustrates a method for providing a new regimen to a person/group/company to prevent adaptation or loss of a chronic effect using a regular program. The closed loop system is subject/group of subjects/company-tailored and has a continuous or semi-continuously learning-closed loop system, providing a new regimen per session and within the session.

FIG. 5 schematically illustrates three types of task tables: A table for work tasks is sorted by the levels of concentration/efforts/resources/energy required for their execution. Each type of task is also prioritized, and specify the amount of time required for its accomplishment; A table for extra work activities includes sports, music, meals, massage, etc.; and A table for “do nothing periods” is part of the daily schedule where no task is allocated.

FIG. 6 schematically illustrates four types of meters: A personal meter where the individual, the team and/or the company specify their concentration level, efforts, energy level, and resources they anticipate for each period during the day; A performance meter that measures the efficiency and overall goal achievement; A variability meter that measures the degree of variability in an individual, team, or company; A work environment meter is based on inputs from every team member and the management.

FIG. 7 schematically illustrates a calendar based on the algorithm using the task tables and the meters. Using the color code shown in FIGS. 5 and 6, an example of a calendar prepared by the algorithm is shown. In this example, the algorithm inserts the tasks based on the tables and matches them together based on the different levels of the algorithm used.

FIGS. 8A-B schematically illustrates a subject/group of subjects/company-tailored, continuously developing, randomization-based algorithm for preventing or slowing aging processes. Introducing noise can serve as a measure for delaying aging processes. Regular physical activity (PA) promotes mental and physical health. The association between intra-individual variability in PA and disability among non-athlete adults was tested. The figure shows the association between intra-individual variability in PA frequency with physical and cognitive disability: FIG. 8A shows the disability rates at the end of follow-up (left) and change in cognitive function by the end of follow-up (right) among the entire study cohort, across high and low sport intensity at baseline. FIG. 8B Shows the same analysis as in FIG. 8A, among the subset of and end of follow-up.

### DETAILED DESCRIPTION

In the following description, various aspects of the disclosure will be described. For explanation, specific configurations and details are outlined to provide a thorough understanding of the different aspects of the disclosure. However, it will also be apparent to one skilled in the art that the disclosure may be practiced without specific details being presented herein. Furthermore, well-known features may be omitted or simplified so as not to obscure the disclosure.

According to some embodiments, there are provided herein algorithms, methods, devices, and systems for improving efficiency and function in a workplace and any type of life activity, including creating artworks and preventing and/or slowing aging processes by implementing patterns of intra and inter-subject and/or group of individuals and/or companies' variabilities, with and without combining them with non-variable individualized patterns, with or without adding patterns learned from other subjects, teams or companies, that are identified from the human body as part of the inherent variability/variabilities of any other body network or signature and/or artificially added variability patterns.

According to some embodiments, there are provided herein computerized systems and methods for improving function of a subject, a group of subject, a team, a company, by introducing variability into work, for overcoming burnouts and more of the same problem, improving efficiency, preventing and/or slowing aging processes, and identifying, quantifying, and implementing at least one inherent variability pattern which is based on patterns learned from a specific subject and/or group of subjects and/or companies.

According to some embodiments, there are provided herein devices, systems, and methods for real-time or delayed altering of the parameters of the system's regimens, for improving functions and regimens, or devices intended for improving performance, and for preventing and overcoming burnouts in subjects/teams/companies.

According to some embodiments, there are provided software, devices, systems, and methods for real-time or delayed altering of the parameters of regimens to improve the regimen's long-term effect. According to some embodiments, any regimen and/or device-generated maneuver and/or stimulation, wherein the parameters are updated within the period, for personalizing the regimen parameters to a subject/team/company and increasing the efficacy of the regimen for achieving the desired goal, and to prevent long-term adaptation. Any system used by humans or affects human function, wherein the parameters are updated using inherent variabilities signatures and/or other types of variability with and without other individualized patterns from a subject or other subjects or companies, can increase the system's efficacy for achieving the desired goal. Output parameters may be continuously, semi-continuously, or conditionally updated based on measurements and inputs provided to a compute circuitry configured to facilitate closed-loop machine learning capabilities.

According to some embodiments, there are provided herein devices, systems, and methods for using identified patterns of variability to improve functions and performance.

According to some embodiments, there are provided herein devices, systems, and methods for quantifying these parameters and generating number(s)/factor(s), which can be implemented into a functional regimen/an operating algorithm for improving the function.

According to some embodiments, out of these variability patterns, an individualized/team-derived/company-derived pattern-based number(s)/factor(s) are generated and implemented into the operating method of the system. For example, the ratio between two heart beat variabilities from two consecutive beats; mean of ratios between consecutive variabilities in heart rate; ratios between two or more breathing variabilities; any sample entropy algorithm; use of a complexity index; multiscale entropy measurements; methods used to quantify nonlinear or chaotic systems; using the ratio between differences between cells in gene expression, proteomics or metabolomics at a level of single cells or multiple cells; or any combination of these; are implemented into operating systems aiming at improving the function, or into algorithms which control the function, or of any device or method being used by humans, or for humans to improve function such as stimulating device of any type.

According to some embodiments, any output of function of any type of anti-aging system or device or activity, and any type of timetable, calendars, solutions for setting up goals in workplaces, and selection of future goals, which can be changed, function of devices used by humans for any purpose whether personal or non-personal, algorithms designed for improving function, can be improved by implementing parameters which are based partially or solely on random variability patterns, semi and pseudorandom parameters, and inherent types of variability patterns from the user or other users, and can be combined with additional parameters whether relevant or irrelevant to the function of the system, including individualized and non-individualized parameters which are not variable, any type of body networks, combining them with parameters from other subjects or other systems, for achieving the desired goal or for improving the functionality of a personal and workplace calendars, timetables, goals design methods, performance monitoring methods in a dynamic-personalized way.

According to some embodiments, the parameters are determined and updated using a machine learning system. It provides parameter values based on feature values received from and/or related to the user and to the performances of an individual, a team, or a company of any size. According to some embodiments, the machine learning system may be a deep learning system in which learning on some features is guided or supervised while learning on others is unguided or unsupervised. According to some embodiments, the number of layers/levels of deep machine learning depends on the number of features.

According to some embodiments, the user updates the machine with progress towards the target goal, which is set by the subject by a team or by a company, for improving the function towards a required level for overall performance improvement. The learning machine provides updated functional regimens-relevant parameters and regimens, and/or stimulation or other device-related parameters based on data learned from the target system function and/or subject, team, or company performance.

According to some embodiments, the user updates the machine with related or non-related inherent variability parameters with or without additional biomarkers or parameters, or combinations of regimens and/or user and/or other users that may be given to other users with similar feature values such as performance, scores related to the target function, conditions and so on, and data specific to the user, for example progress towards improving the function reaching pre-defined or newly defined goals, overcoming burnouts in work, improving performance of an individual, a team and/or a company and slowing aging processes.

According to some embodiments, user inputs may include all physiological or pathological parameters, personal, group-related and/or company-related and environmental parameters relevant directly or indirectly to the target and/or to regimens or procedures. These parameters may be relevant to a subject, specific regimen, or system and not necessarily to all subjects. The input parameters may involve one or more patterns of inherent variability from different levels in the subject body and/or from other subjects. These parameters may be used independently or combined with other parameters from artificially added randomness parameters.

According to some embodiments, any quantification method or methods can be used for generating number(s)/factor(s) or any other method for quantifying the pattern(s) of variability and combining them with non-variable parameters and with any other type of parameters, signatures, or networks, and implement them into the function of a system, or into an algorithm to be used for improvement of the function of systems. These include any entropy type of algorithms, indexes used for analyzing irregularity, methods for quantifying networks, methods for quantifying chaotic and non-linear systems, and the like.

According to some embodiments, the user may update the machine, or the machine may receive inputs from the user and/or from other users to inform the algorithm in a way that enables to redirect or further define the function of the algorithm or the device, or the treatment regimen, or the function of an engine, or the performance of a system, to the user, following a closed-loop system.

The data received in real-time or not, is continuously analyzed based on one subject's and/or a group of individuals and/or companies of variability, and/or subgroups of subjects, system function parameters, and biomarkers which are directly and indirectly associated with the system, the subject, the group and/or the company, subject, group, and/or company performance, system performance, and data from other subjects, groups, and/or companies.

As used herein, the terms “learning machine,” “update module,” and “update system” are interchangeably used and refer to an integrated or communicatively linked component of the system, which is configured to receive input data in the form of user data, or any variability pattern, define a method to quantify the input, and has the option to combine it with data from the subject or other subjects which is not necessarily based on variability patterns. These patterns or parameters are directly or indirectly associated with the system's function and can include features that measure directly or indirectly relevant human indications. The learning machine can implement the data from these patterns into functional parameters of the system or into an algorithm that impacts the function of the target system to reach an endpoint predefined by the user, the system, or other subjects.

According to some embodiments, the input data on the user, along with the input received from other users on a continuous or semi-continuous basis, is processed by the controller, which is based on a closed-loop system that continuously evaluates the distance of the tested parameter from the level to be achieved or the direction and/or rate of changes in the physiological or pathological measurement/parameter, generates an improved algorithm being transformed into new output.

The output may be in the form of an input into a calendar, a time table, any software or method for dealing with arranging times, tasks, or planning, and/or as an alert delivered to the subject, group of individuals, and/or a whole company or to a device, an engine, a system associated with humans, via a cell phone-based application, or by any other method, which will alter the time for performance of a task, and/or a function of the target, or the function regimen of the target for improving their function.

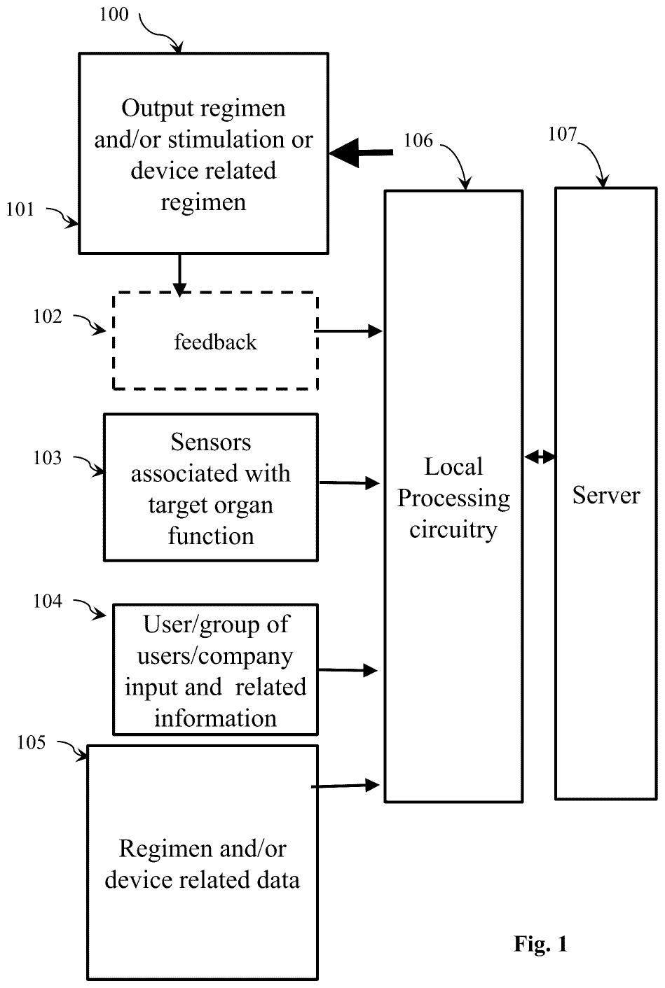

Reference is now made to FIG. 1. According to some embodiments. System 100 includes a challenged-functional regimen alert output device and/or maneuver/stimulator 104, which is configured to provide function regimen alert output to a target to achieve a desired goal as determined by the user, or by others, or a physiological effect for improving function, optionally one feedback mechanism 103 associated with function output and/or maneuver/stimulator 104, configured to provide measurements of functional indicators relevant to target system function or any other system related or non-related parameter, or technical information related to the system. These parameters may be related, or indirectly related, to the target system, which the method or algorithm aims to improve its performance.

According to some embodiments, system 100 includes additional external sensors 101 and 102, which can receive or identify inherent variability patterns to be incorporated into the output after being processed by a processing device 105, which uses one or more methods for quantifying these patterns. These include methods for quantifying networks and quantifying chaotic systems and non-linear systems. For example, variability patterns of pulse, rate of breathing, and oxygen saturation, which provide data on overall body function, can be implemented with the other inherent variability patterns, such as gene networks or protein-based variability patterns, of the subject, for generating number(s)/factor(s), which are used for improving the function of the subject/group of subjects/company. The information from the feedback mechanism 103 is provided to the local processing circuitry, configured to control the operation of 104 based on inputs that include measurements of external or internal sensors 101 and 102 and optional feedback mechanism 103.

According to some embodiments, a user may be instructed or advised to measure their function and/or regimen and/or target-associated biomarker periodically or provide an inherent variability pattern periodically or continuously, aiming to achieve the goal at certain times or after/at/before certain events. According to some embodiments, processing circuitry 105 may communicate with a remote server 106 to tap into the computing performance and/or data of previous/other users. According to some embodiments, remote server 106 may be a cloud computer. According to some embodiments, the processing circuitry is designed for a continuous or semi-continuous closed-loop data input and output, wherein algorithm output and/or device-generated maneuver or stimulation parameters are adjusted based on the input information and data.

According to some embodiments, the output algorithm may be introduced by a new set of tasks and/or function regimen and/or by a device-generated maneuver. It may be introduced to provide an alert for a preferred function regimen to achieve the desired goal.

According to some embodiments, the algorithm can incorporate data generated from other subjects and identify the preferred variability patterns and/or other patterns based on which to determine a subject's group and/or company-tailored irregularity in the function of a system.

According to some embodiments, the algorithm can also identify other methods for quantifying the patterns of variability such that a better end response is noted. The number(s)/factor(s) generated from one or more of the inherent individualized, group-related, and/or company-related patterns are incorporated into the operating system, which, for example, determine how to design a calendar, a timetable, a task table, any planner, for an individual, a group, or a company, and design and/or use of any anti-aging treatment, device, or regimen. These numbers generate a personalized and/or group and/or company-type irregularity of the function or any alteration of the operating system to improve its function. Irregularity is a random perturbation with the power of randomness, which is exploited to enhance functions.

According to some embodiments, an alert is delivered via a cloud-based alert system, for example, in real-time, such that the alert is connected to any partial/complete loss of an effect of regimens and/or device treatment or maneuver.

### EXAMPLES

### Example A: Three Types of Task Tables

a. A table for work tasks sorted by concentration levels/efforts/resources/energy required for their execution. Each type of task is also prioritized, and specify the amount of time required for its accomplishment.
b. A table for extra work activities includes sports, music, meals, massage, etc.
c. A table for “do nothing periods” that is part of the daily schedule where no task is allocated.

### Example B: Four Types of Meters

a. A personal meter where the individual, the team and/or the company specify their concentration level, efforts, energy, and resources they anticipate for each period during the day.
b. A performance meter that measures the efficiency and overall goal achievement
c. A variability meter that measures the degree of variability in an individual, team, or company.
d. A work environment meter is based on inputs from every team member and the management.

### Example C: Four Levels of the Algorithm

Level 1 is an open-loop system that matches in a random manner tasks from the work task-table into the calendar based on priorities and time required for them, and the level of concentration and effort required. It can also change tasks between workers randomly.
Level 2 is an open loop system that introduces extra work tasks and randomly “does nothing period” into the calendar to match the period pre-defined by the worker, the team, and management.
Level 3 is a closed-loop system that adapts the variability in inserting the task to performance according to the performance meter. It is done using an algorithm that personalizes the variability of matching the tasks to the individual, the team, and/or the company.
Level 4 is a closed-loop system that quantifies and introduces measures of variability into the algorithm, e.g., a low heart rate variability period leads to a high rate of variability in the tasks. Any stimulation techniques that incorporate variability measures are included. It also considers outputs from the variabilities of the tasks themselves.

### Example D: A Calendar Based on the Algorithm Using the Task Tables and the Meters

Using the color code shown in examples A and B and the algorithm in C, an example of a calendar prepared by the algorithm is shown. In this example, the algorithm inserts the tasks based on the tables and matches them together based on the different levels of the algorithm used.

If the personal, group, and/or company meters show that they are in the green zone, highly concentrated, and highly efficient, the person and team can move on and not stop. The meters are always dynamic, and the algorithm continuously adapts the tasks to the calendar.

It moves the tasks during the day based on the person's ongoing insight into his efficiency status. Tasks are divided according to the degree of concentration they require into those that require complete concentration, tasks that are more technical and can be done almost automatically, and dull. The person lists his tasks based on colors. If the subject's meter shows he is in the light red, the algorithm will select one of the tasks for that period which do not require full concertation. It prevents wasting the green periods on reddish tasks. The person can use his time more effectively. It prevents him from wasting his green time on tasks that can be done during light-red periods. Similarly, it prevents forcing the person from doing deep green tasks that require the best out of him during the reddish periods.

It is fully personalized to an individual, a team, and a company. Colors change all the time. A person may hear a good joke or have a light snack that will move him from the red zone into the green one. Tiredness, not directly related to the number of hours of sleep, feeling burned out, over-chronicity, and adaptation can all push him in the less efficient direction.

Using a one-hour task list is an ideal scheme. Some tasks may require two hours; some may require three hours. Others are 30 minutes' tasks. The software has the personal tasks divided into several columns based on the colors and subdivided into time categories. The tasks are moved into the calendar based on the efficiency meter. At the end of each day, the person and/or team leader and/or company manager prepare the list and fit the boxes of tasks into the times on tomorrow's calendar. He can anticipate 2-3 green hours in the morning and two in the afternoon. The software fits the red tasks into the rest of the day. If tomorrow is discovered to be a greenish day for the person, he can change it along the way, and vice versa if it turns out to be a reddish day.

During a workday, the subject, team and/or the company can continuously receive tasks from above, below, or add some personal tasks. These are added to the table based on the concentration each task requires and the appropriate color assigned. The software automatically adds the tasks to the free opening in the calendar based on the colors. Priorities can also be administered when added to the table. The colors are personalized to fit each subject's clock, such that morning people will have more green slots in the mornings, and evening people will have more green periods.

### Example E: Biosensors-Based Personal Efficiency Meter

The personal meter can be connected to any personal biosensor, such as a sensor that measures heart rate, heart rate variability, oxygen saturation, of any other type of biosensor that measures any physiological or pathological parameters. These can also include invasive tests, revealing the person's status in a certain period. Connecting the personal efficiency meter to glucose levels in patients with diabetes can improve the meter's accuracy in a personalized way. The parameters measured can be measured periodically or continuously. A chronobiology-associated gene of proteins can be used to improve the accuracy of the personalized efficiency meter. Any energy assessment, including subjective assessment of an individual's energy, a group of individuals, and/or a company, can be associated with the personalized meter. It means that if the person knows he has more energy at a certain period, he can turn that period to green, and the software assigns a green task.

### Example F: A do-Nothing-Based System for Overcoming Burnout Periods

A way to improve performance in the red zones can be to add a “do nothing period.” It is an hour marked in white of doing absolutely nothing. It may be much more helpful than a continuous attempt to fit a green task into a deep red time slot. The “do nothing hour” can become much more efficient to enable a subject to accomplish much more. Do nothing is not listening to music, reading the newspaper, talking, or eating. It is doing nothing, and no task is designated for this period.

This box allows the individual, the team, and/or the company to allocate times when they are not required to do anything. It can be planned if a person anticipates such a day in front of him or can be decided during the day when it seems to him that one hour of doing nothing is what he needs. The do-nothing can be half an hour of reading, walking outside, talking to a colleague or a friend, or just taking a short nap.

### Example G: A Noise-Based System for Overcoming Burnout Periods and Improving Efficiency

The table where the tasks are listed has a prioritization system. However, if two or more tasks in the same color have the same degree of priority, the software will randomize them. It implies that a pseudo-random number generator is used to select the type of task. The selection can also be randomized based on any biosensor or other personal or non-personal input data. Noise-based software can incorporate parameters based on biological noise, such as heart rate variability or any other type of biological noise.

The system enables overcoming burnout and the problem of doing more of the same. If a person keeps achieving what he is required or wants from himself, is satisfied with it, and enjoys the way to achieving it, he can keep using the same planner. However, doing more of the same in many cases leads to plateaus. Using methods, formulations, and adapting newer behaviors helps. However, they may often end up in some plateau. For some, it is simply about being burned out.

In some cases, the primary cause of being burned out is the feeling of standing in the same place. The software can overcome plateaus by introducing variabilities in tasks. The changes are introduced randomly based on the random selection of tasks or any input data.

### Example H: A Subject/Group of Subjects/Company-Tailored, Continuously Developing, Randomization-Based Algorithm for Preventing and/or Slowing Down Aging Processes

Introducing noise can serve as a measure for delaying aging processes. Regular physical activity (PA) promotes mental and physical health. The association between intra-individual variability in PA and disability among non-athlete adults was documented. Non-disabled adults aged>50 followed over 14 years. Self-reported PA frequency was documented bi- to triennially. Low PA intensity was defined as vigorous PA frequency less than once a week. Stable PA was defined as an unchanged PA intensity in all consecutive middle observations. The primary outcome was defined as a physical limitation in everyday activities at the end of the survey. Secondary outcomes were cognitive functions, including short-term memory, long-term memory, and verbal fluency. The study included 2,049 non-disabled adults with a mean age of 53 and 49.1% women in the initially low PA intensity group; variability was associated with a reduced physical disability (45.6% vs. 33.3%, stable vs. unstable PA; P=0.02; adjusted P=0.03). Among individuals with the same low PA intensity at the beginning and end of follow-up, variability was associated with reduced physical disability (56.9% vs. 36.5%, stable vs. unstable PA; P=0.02; adjusted P=0.04) and improved short-term memory (score change: −0.28 vs. +0.29, stable vs. unstable PA; P=0.05). The data suggests that incorporating variability into PA regimens of inactive adults may enhance their physical and cognitive benefits. The figure shows the association between intra-individual variability in PA frequency with physical and cognitive disability: (A) shows the disability rates at the end of follow-up (left) and change in cognitive function by the end of follow-up (right) among the entire study cohort, across high and low sport intensity at baseline. (B) Shows the same analysis as in A panel, among the subset of and end of follow-up.

### Example I: A Variability-Based Software for Continuous Design and Developing New Artworks

In this example, a subject can take any artwork, such as an impressionistic work, and use software to virtually cut it into pieces and then randomly recombine it. The outcome can be more admirable and more meaningful. The software can change the order of the pieces continuously such that the screen will keep showing different pictures every period.

The embodiments show that individualized, team-related, and/or company-based regimens of irregular challenged-work tasks and anti-aging maneuvers, or any maneuver, procedure, or stimuli regimen that is subject and/or company-specific and/or task and/or goal-specific can continuously improve performance, prevent or overcome adaptation, slow aging processes, enabling achieving a better effect within a shorter period, and with better adherence. The disclosed algorithm is subject and/or company-specific, performance-specific, genetic and task-specific, and goal-specific.

According to some embodiments, the algorithm or deep machine-learning algorithm can benefit by learning from large numbers of subjects and companies with the same tasks and enable tailoring of the solutions to be more beneficial for certain subjects, groups, and/or companies. A cell phone-based application, or any other mode of alert system, monitors subject and/or company capabilities and degree of variability measured by biosensors or any other type of measurement and matches the tasks to predefined periods. A performance meter provides input to the system. The dynamic algorithm introduces variability in the tasks based on their pre-defined level of required capabilities and prioritizations, extra work activities such as sports, music, meals, and “do-nothing periods.”

According to some embodiments, there are provided herein devices, systems, and methods for the generation of algorithms in a way which subject and/or company-tailored, task-tailored, goal-tailored, open or closed-loop continuously or semi-continuously changing the tasks for prevention or overcoming of adaptation and burnouts and for overcoming partial or complete loss of productivity, via altering any of the parameters associated with the said tasks or maneuvers, by any change which is of relevance for improving the long term effect, or for continuously achieving a better level of performance. It can also be achieved using devices that generate maneuvers or stimulations. The algorithms and/or the algorithm-based devices can be used for continuous prevention or overcoming of adaptation or loss of a maximal effect of all types of burnouts and prevent the loss of performance and aging processes.

According to some embodiments, any task-related maneuver, wherein the procedure parameters are updated within or between the working periods, for personalizing the algorithm parameters and for increasing the accuracy and/or efficacy of the maneuver for continuously achieving a better function, and to prevent adaptation continuously or for ensuring prolonged maximal effect on overall performance.

According to some embodiments, the algorithm that provides new regimens is subject-specific, function-specific, company-specific, task-specific, goal-tailored, and/or maneuver-tailored, which is based on random alteration of the tasks by providing a specific regimen to a certain period using an algorithm-based alteration of tasks regimens and/or algorithm-based alteration of anti-aging procedures, and/or stimulating or other device or maneuvers used for overcoming adaptation.

According to some embodiments, the algorithm that provides a new task or performance regimen is subject-tailored, group and/or company-tailored, and/function-tailored, and/or topic-tailored, disease-tailored, and/or maneuver-tailored and/or aim program-tailored, is based on alteration of the regimen, by subject/group/company/performance/topic/physiological aim-tailored continuously or semi-continuously, and developing randomization-based algorithms for improving the performance and prevent and/or slow aging process. This method overcomes adaptation to monotonic regimens, including those that involve a gradual increase of difficulty and/or gradual increase in change or the use of regular break sessions.

According to some embodiments, the algorithm exerts a positive burden on the brain-target connections, further improving performance, thus preventing adaptation and increasing efficacy and task achievement, and preventing or delaying aging by improving the effectiveness of anti-aging maneuvers or therapies.

According to some embodiments, the algorithm that provides a new variability-based regimen is subject and/or team and/or company-tailored, function-tailored, and/or task-tailored and/or physiological aim-tailored and/or device-generated maneuver-tailored by using a method for continuously or semi-continuously improving the ability to reach a better result, including an ability to reach better endpoints by using a non-gradual, non-stepwise, approach in a subject/task/topic/environment-tailored approach. This method improves performance within a shorter period, with less adaptation and higher adherence.

According to some embodiments, the subject and/or team and/or company-tailored continuously developing randomization-based algorithm provides new time planners, calendars, tasks managers' regimens which are subject/team/company-tailored and/or performance-tailored, and/or performance-tailored, and/or task-tailored and/or physiological aim-tailored and/or anti-aging-tailored, and/or maneuver-tailored by using a method to prevent or ameliorate any burnout, overcome more of the same problem, and aging processes, lack of adherence to any work or tasks and others.

According to some embodiments, the parameters are determined and updated using a machine learning system, which provides parameter values based on feature values received from and/or related to the user.

According to some embodiments, the machine learning system is a deep learning system in which the learning on some features is guided learning while learning on other features is unguided.

According to some embodiments, the number of layers/levels of deep machine learning depends on the number of features or associations between them.

According to some embodiments, the user updates the machine with inputs indicative of progress towards the targeted physiological goal, and the learning machine provides an updated method of challenged-task performance tool, timetable, task manager, calendar or anti-aging maneuver, regimen or maneuver administration according to the tailored parameters relevant for the said subject/team/company procedure based on data learned from the user and/or other users.

According to some embodiments, as used herein, the term goal or target may refer to value, gradient, or change in any measure or parameter in a desired direction or achieving a better score in a task reflecting, for example, the goal may be avoiding the development of adaptation to task aimed at improving the ability to perform better. In this case, such a goal may be avoiding tolerance to a specific regimen by setting a deep-machine learning open or closed-loop individual based-algorithm that sets a new regimen for the subject/team/company. The new regimen is designed with or without setting a specific range as a parameter/value change target.

According to some embodiments, a user may update the machine, or the machine may receive inputs from the user and/or other users that are being used to update the algorithm in a way that enables redirecting or further defining the changes in the regimen via providing of a change in one or more of the parameters which are relevant to the said regimen and to the said subject. The learning machine provides updated parameters based on continuously or semi-continuously learned data from other users. The data received is continuously or semi-continuously analyzed based on sub-groups of subjects, including based on task regimens function-related parameters, targets to be achieved, subject/team/company-related parameters such as age gender, and other factors which are subject/team/company and/or aging and/or overall aim of regimen-related.

According to some embodiments, a mobile phone-based system, or any other type of alert system, is provided for dispensing instructions to subjects, including an update module computationally configured to receive a plurality of feature values and provide the relevant parameters for setting a new working regimen. These parameters may be changed based on the type of input received from the said subject, including measurements of physiological parameters such as results of tests in a said topic, achievements in competitions, pulse, respiratory rate, oxygen consumption, and information received from EEG, ECG, EMG, MRI, CT, PET, PET/CT, US, X-ray, DEXA, blood tests, any physiological or pathological biomarkers, parameters which are directly or indirectly related to performance and/or to the subject.

According to some embodiments, the processing circuitry of the update module is operated to facilitate machine-learning capabilities, wherein supervised and/or unsupervised learning is utilized.

According to some embodiments, the machine learning capabilities include deep learning capabilities.

According to some embodiments, the physiological goal is avoiding development and/or overcoming adaptation, habituation, or tolerance to long-term working tasks and anti-aging maneuvers.

According to some embodiments, the machine learning success factor is maintaining physiological change and/or improvement in target function.

According to some embodiments, the features of the machine learning are selected from a list including the type of regimen used, type of maneuver, type of target, performance/final aim to be achieved, working environment, mode of administration of the regimen, and subject/team/company-related parameters including performance on previous tasks, performance of previous competitions, age, weight, gender, ethnicity, geography, pathological history/state, temperature, metabolic rate, glucose levels, blood tests and any physiological or pathological parameters that can be measured whether directly or indirectly associated with the aim of task or with the individual/team/company or with the physiological target; any biomarker which is directly or indirectly associated with the regimen, and/or with a subject and/or a group of subjects and/or companies.

According to some embodiments, the parameters are determined and updated using a machine learning system, which provides parameter values based on feature values received from and/or related to the user.

According to some embodiments, the number of layers/levels of deep machine learning depends on the number of features or associations between them.

According to some embodiments, the user updates the machine with inputs indicative of progress toward the target effect goal, and the learning machine provides an updated algorithm and/or device-derived parameters based on data learned from the user and/or other users. In contrast, a different physiological goal may be given to other users with similar feature values, such as function, performance on tests, and scores that are of relevance to performance, race, age, gender, health conditions, and so on, as well as data specific to the user.

According to some embodiments, the newly generated regimen further contributes to progression towards a target goal, and the learning machine provides updated algorithm and/or procedure parameters based on data being continuously or semi-continuously learned from the user and/or other users.

According to some embodiments, the data is continuously or semi-continuously received and is analyzed based on factors associated with the target function, the overall aim to be achieved, performance of previous tests, scores achieved in previous competitions, status of other tasks, and/or subgroups of subjects, targets of physiological levels to be achieved, and biomarkers which are of relevance to the regimen.

According to some embodiments, the loop will include a maneuver/stimulation device, including a maneuver/stimulation inducer, configured to generate a regimen based on stimulation parameters to affect a change in targets or tasks.

According to some embodiments, the system includes a communication unit configured to allow the transfer of data to the central part of the algorithm, which sets up the output and/or a signal to a maneuver/stimulation device for modifying one or more of the regimen parameters and/or maneuver/stimulation parameters, an update module, including a processing circuitry, configured to: obtain a signal from the sensor, determine the algorithm and/or maneuver/stimulation parameters based on the signal obtained from the sensor, provide an alert to the new regimen and/or provide the device with the determined maneuver/stimulation parameters via the communication unit.

According to some embodiments, the processing circuitry of the update module is operated to facilitate machine-learning capabilities, wherein supervised and/or unsupervised learning is utilized.

According to some embodiments, the algorithm is provided for continuously achieving a desired change, and the learning machine's success factor is achieving, maintaining, and continuously improving this physiological change.

According to some embodiments, the goal is a continuous prevention of adaptation or partial/complete loss of an effect when performing any task or regimen and prevention of attenuating aging processes in healthy subjects and for subjects with any pathological condition that requires improved function, or conditions in which a subject wish to improve performance or any of the conditions comprising the same.

By setting up a continuous irregularity within a specific range predetermined for each subject/group/company and for each task or regimen and/or maneuver being used.

By continuously setting up a new regimen and/or a new device-related signal, with an irregularity within a specific range predetermined for each type of regimen and each subject/group/company.

According to some embodiments, the sensor is configured to measure mental function, any physical activity, any score on a test, any performance in a competition, temperature, oxygen levels, blood pressure and/or blood tests, organ activity, and/or any physiological or pathological parameters or biomarkers, that can be measured whether directly or indirectly-associated with the target of performance.

According to some embodiments, there is provided a regimen and/or a device that is aimed at targeting said task, inducing devices that provide stimulation for the brain, abdominal stimulation, or any organ stimulation, whether this organ is associated with the target of the regimens, or with the target or not, including a maneuver/stimulation device inducer, configured to generate a stimulation action based on parameters that affect a change in a task, and a communication unit, configured to allow transfer of data between the device and an update module, wherein the update module includes processing circuitry, configured to obtain a signal from at least one sensor indicative of a physiological or pathological property, determine algorithm and/or maneuver/stimulation parameters based on the signal obtained from the sensor, and provide the device with the determined algorithm/maneuver parameters via the communication unit.

According to some embodiments, a method for a continuous/semi-continuous/non-continuous/conditional, open or closed-loop for any maneuver/stimulation or modification, including providing/placing in the proximity of a body part a modification/stimulation-device, or any device which can provide any signal, or can induce any direct or indirect change in function, or transplanting a maneuver/stimulation-device, with a maneuver/stimulation inducer, providing initial parameters to the device, based on initial acquired information and a desired change in function, providing maneuver/stimulation via the inducer, or providing any signal or effect that can alter function, based on initial stimulation parameters, obtaining information from the user and/or device or other sources, and updating the relevant task and procedure parameters based on the obtained information.

According to some embodiments, a method for a continuous, semi-continuous, conditional, or non-continuous closed-loop for generating new regimens by providing an alert for the specific algorithm-dependent parameters, time, degree of difficulty, or any other task-related parameter.

According to some embodiments, continuously updating the newly-generated regimen based on alteration of any of the said regimen relevant parameters and/or device-associated parameters, including, for example, degrees of difficulties, times for performance of the task, combining several types of tasks or procedures, and/or using stimulation parameters, includes utilizing machine learning capabilities. According to some embodiments, the machine learning capabilities include deep learning in a closed-loop method.

According to some embodiments, the machine learning capabilities are configured to be operated on a set of features by receiving values. According to some embodiments, the output new regimen is provided to the subject by a cell phone, computer-based alert system, or any other type of alert system, and/or by a maneuver/stimulation device, including a wearable/implantable device. According to some embodiments, the stimulation device or the device that affects function is configured to be swallowed by a user. According to some embodiments, the device is configured to be placed on the user's body or used via any device in direct or indirect contact with the human body or the target.

According to some embodiments, a goal is improving function by preventing adaptation or loss of an effect or non-responsiveness to regimens and/or improving performance.

Certain embodiments of the present disclosure may include some, all, or none of the above advantages. One or more technical advantages may be readily apparent to those skilled in the art from the figures, descriptions, and claims included herein. Moreover, while specific advantages have been enumerated above, various embodiments may include all, some, or none of the enumerated advantages.

In addition to the exemplary aspects and embodiments described above, further aspects and embodiments become apparent by referencing the figures and studying the following detailed descriptions.

The non-obviousness of some of these embodiments comes from the fact the methods utilize an algorithm which is subject and/or group of subjects, and/or companies-tailored and/or task-tailored, and/or performance-target to be reached in the said function-tailored, by a way of altering the regimen and/or time and/or method of administration and/or combination of different tasks, and/or combination of different degrees of difficulties and/or combination of procedures which targets and/or use of maneuver/stimulation to any organ and/or by using devices and/or any type of regimen as an adjuvant to the main task, for improving the function of a said subject/group/company, for improving overall capability of a subject/group of subjects/company, for prevention/treatment of adaptation to regimens, or as sole maneuvers, and for prevention and/or slowing of aging processes of any type, are not expected based on the current knowledge of subject/team/company function, and chronic therapies/regimens.

According to some embodiments, the input data on the user/team/company, along with the input received from other users on a continuous or semi-continuous basis, is being processed by the controller, which is based on a closed-loop system that continuously evaluates the distance of the tested parameter from the level to be achieved or the direction and/or rate of changes in the physiological or pathological measurement/parameter, generates an improved algorithm being transformed into new output.

The output can be an alert delivered to the subject via a cell phone-based application or any other method, instructing the subject/group/company on the regimen or parameters relevant to the maneuver.

According to some embodiments, the output that a generated procedure or stimulation inducer can deliver is configured to affect a maneuver/stimulation by providing a mechanical, magnetic, electrical, temperature-based, ultrasound-based, or any other type of signal or other maneuver generated by the device to the target body part or any other body part, by physical movement, using various types of rate and rhythms of stimuli with various frequencies, amplitudes, durations, and intervals, in structured or random manner, or other types of direct or indirect stimuli.

According to some embodiments, reference is now made again to FIG. 1 of an output alert and/or maneuver/stimulation system 100. According to some embodiments, system 100 includes a regimen alert output device and/or maneuver/stimulator 101, which is configured to provide regimen alert output and/or device-generated procedure or stimulation to achieve a desired effect for improving function, optionally one feedback mechanism 102 associated with task output and/or stimulator 101, configured to provide measurements of physiological indicators relevant to target function or any other task-related or non-related biomarker, or technical information related to 101, such as battery charge level. These parameters may be related or indirectly related to the target the algorithm aims to improve.

According to some embodiments, system 100 further includes additional external sensors 103, for example, pulse, rate of breathing, oxygen saturation, blood tests that provide data on the task function or overall body function, or any other test and the like, or any performance of the subject/group/company, which along with the information from feedback mechanism 102 are provided to a local processing circuitry 102 which is configured to control the operation of 101 based on inputs that include measurements of external or internal sensors 103, and optional feedback mechanism 102. According to some embodiments, processing circuitry 106 is further configured to obtain inputs of user-related information 104 and other user inputs, including regimen and/or device-related data 105, based on which the algorithm and/or device output parameters are determined.

According to some embodiments, external sensors 103, maybe regimen-related biomarker sensors, configured to provide local processing circuitry 106 with information indicative of the target task function and regimen-target parameters of the user at certain times. According to some embodiments, a user/group of users/company may be instructed or advised to measure their task function and/or regimen and/or task-associated biomarker periodically or any other parameter that may directly or indirectly relate to achieving the goal at certain times or after/at/before certain circumstances.

According to some embodiments, processing circuitry 106 may communicate with a remote server 107 to tap into the computing performance and data of previous/other users. According to some embodiments, remote server 107 may be a cloud computer.

According to some embodiments, the processing circuitry is designed for a continuous or semi-continuous closed-loop data input and output, wherein algorithm output and/or device-generated maneuver or stimulation parameters are adjusted based on the input information and data.

According to some embodiments, a new regimen and/or a device-generated maneuver may introduce the output algorithm. It may be introduced to provide an alert for a preferred regimen based on task or procedure-relevant parameter changes.

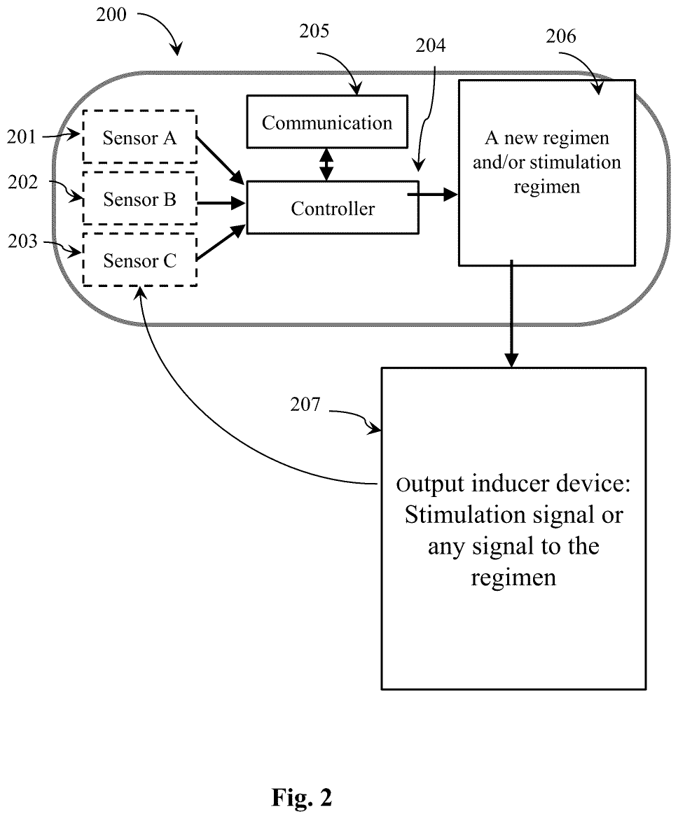

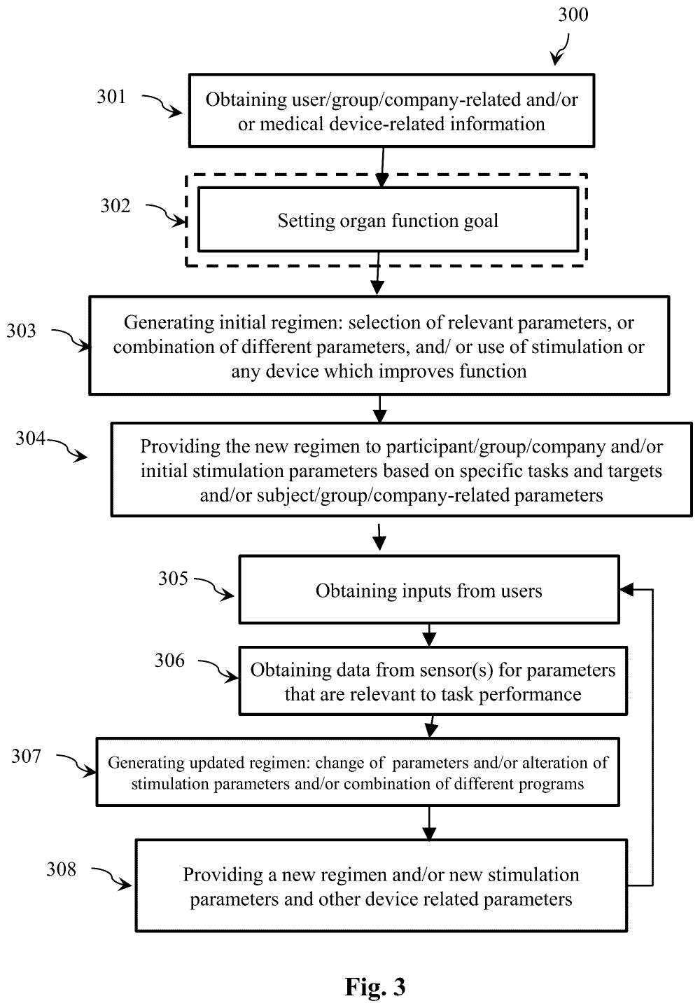

Reference is now made to FIG. 2, which schematically illustrates a functional block diagram of a regimen 200, according to some embodiments. According to some embodiments, regimen 200 is in the form of any task regimen, combination of regimens, and/or use of device-generated maneuvers of stimulation or devices that generate a procedure that improves function. It includes several sensors, 201, 202, and 203, which collect data. It includes subject/group of subjects/company-related data regimen or task-related data using biomarkers or parameters which are related, or not directly related, to function, the desired goal, and the pattern of efficacy of a regimen, configured to provide a sum of data to be used for generation of a preferred regimen and/or a preferred device-generated procedure/stimulation, that continuously prevents adaptation to regular perpetual methods. The controller analyzes the data via a controller device 204 and a communication device 205. New regimens and/or device-generated maneuver/stimulation regimens are being produced by a device that summarizes the data 206.

An output device 207 continuously generates a new algorithm, which is delivered to the subject in the form of a new regimen and/or device-generated maneuver/stimulation regimen for altering the mode of the regimen and/or device function. The output's effect data is being re-collected by the sensors 201, 202, 203 and closing the learning loop.

According to some embodiments, device 200 may optionally further include sensors configured to control the operation of the first challenged-regimen parameter or device maneuver parameter inducer, and several additional such output devices to achieve a change towards a goal, according to regimen and/or device-generated maneuver parameters received via the communication unit, which is configured to be in communication with an external or internal update module/unit/circuitry for receiving the parameters, and sending to that information from the sensors, or other operational information.

According to some embodiments, the output device, which continuously generates new regimens and/or a new device-generated maneuver, may include non-transitory memory for storing regimens and device-generated maneuver sessions to be provided to the user. According to some embodiments, the new regimens and new device-generated maneuvers do not include memory thereon for storing stimulation sessions but are controlled by the update unit for continuously changing the regimens' and maneuvers' parameters whenever such a change occurs.

Reference is now made to FIG. 3, which schematically illustrates method 300 for continuously providing updated parameter generation of an alert for a better regimen or any device-generated maneuver/stimulation signal generated, according to some embodiments.

According to some embodiments, method 300 begins by obtaining user/group of users/company-related information (301) which may be sensor measurements or more general information such as subject/group/company-related, regimen-related, task function-related, biomarker-related, and/or any parameter which is directly or indirectly of relevance to the effect of the regimen or maneuver on performance, such as scores relevant to function, performance of subject/group/company, weight, gender, history and the like or data which is specific for function. A task-specific regimen is determined, an alert is sent to the subjects, and a goal is set (step 302), including target function-related endpoints preventing and/or slowing aging processes.

Accordingly, initial output regimen and/or device-generated maneuver/stimulation parameters are determined (step 303) and provide the participant/group/company with a new regimen and/or maneuver parameters based on specific targets and/or regimen parameters and/or task-related parameters, and subject/group/company-related parameters (step 304). Then, input is provided to the subject/group/company and to the device, which may include updated target function measures, to obtain inputs from the participant/group/company (step 305) and/or obtain data from sensors for parameters that are relevant to target performance (step 306). The updated regimens: change of task parameters and/or alteration of maneuver/stimulation parameters and/or combination of different tasks are generated (307). These provide new regimens and/or new maneuver or stimulation parameters (308), and a closed-loop is generated based on updated parameters and then back to step 305 for new closed-loop-based regimens and/or organ maneuver and stimulation regimens.

According to some embodiments, the system can continuously receive input from internal and external devices or tests, scores, task-relevant performance parameters, blood tests, or from subject/group/company history from multiple subjects, which is being processed according to deep machine learning closed loop algorithms such that relevant data from other users is being applied to the specific subject to optimize the type of regimen. In that way, a subject/group/company-specific algorithm is generated based on input from the subject and relevant data from other users or subjects.

According to some embodiments, the deep machine learning algorithm is designed to have several levels of closed loops built one on top of the other but also function in parallel to enable the generation of an optimized output algorithm and/or output maneuvers/stimuli continuously enabling reaching the target or improving function.

According to some embodiments, the updated system (update module) may have a dual local and network architecture, in which, for example, the local unit/circuitry is in the real-time or short-delay loop with the maneuver device and learns and updates the maneuver or stimulation parameters without involving a higher-level computational circuitry, such as a server or a cloud computer. The update system may include a global/network component, wherein inputs may be received from multiple users, and learning from the data of the multiple users may be applied to the stimulation parameters of individual users.

Advantageously, in such a local-global architecture, the stimuli may be updated in a short/immediate closed-loop using the lower level (local) update module, wherein longer and less immediate closed-loop may update the stimuli using the higher level (global) update module.

The two-stage hierarchical architecture of the update system above is exemplary, and other conceptually similar architectures may apply in various embodiments.

As used herein, “update system” or “update module” refers to a component configured in wired or wireless communication with the stimulation device for setting and amending algorithm-based regimens and/or maneuver parameters.

According to some embodiments, each data parameter is received and analyzed with correlation to the algorithm-based regimen and/or maneuver stimuli generated. Thus, the algorithm can determine the type of data or features most relevant for a specific user/subject that correlates with the physiological target or desired physiological change. This input parameter may not be identical to all users/subjects and may not be identical for the same user/subject regarding different physiological targets, objectives, improvements, or desired performances.

According to some embodiments, the algorithm-based-challenged-regimen and/or the device-generated maneuver/stimulation characteristics may change over time even for the same user with the exact desired change, and even if, and if not, there is a positive physiological change in performance. Such changes in regimens and maneuvers characteristics may be done to avoid the habituation of the user to regimens and device-generated maneuvers and maintain a positive change for continuously improved performance and can prevent and slow aging processes.

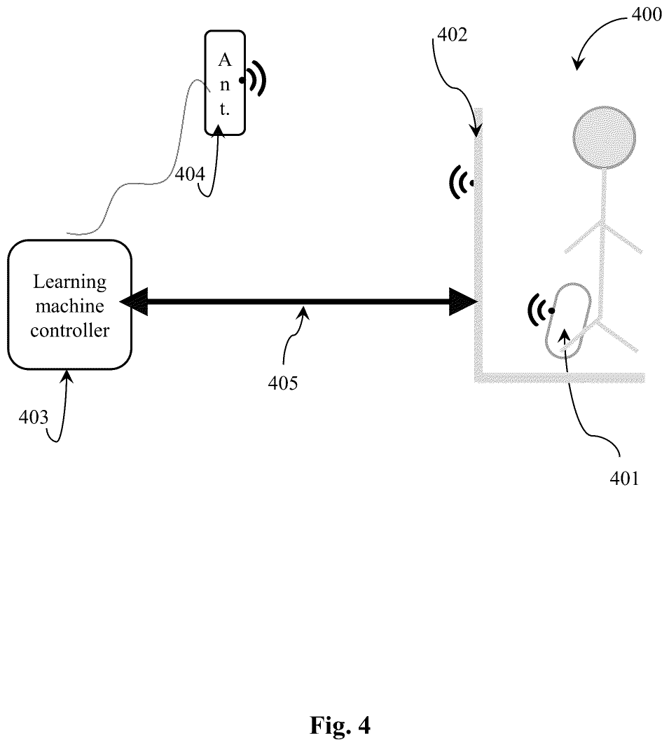

Reference is now made to FIG. 4, which schematically illustrates a subject/group/company performing a task along with a stimulator system 400, according to some embodiments. According to some embodiments, system 400 includes a stimulation device 401, configured to be inserted/introduced to a target area to induce stimulation. Task 402 is connected via a wireless communication link to the control system 403. According to some embodiments, both the task and the stimulation devices are in communication with an update module, such as a continuous or semi-continuous learning machine 403 via a wireless communication link, such as through antenna 404, for sending sensor information from task and stimulation devices 401 and 402 to learning machine 403 and receiving updated algorithm-based new regimen and/or stimulation parameters from there, 405, to adjust the regimen and/or stimulations for achieving desired results towards reaching an improved target goal of a feature and an ongoing improvement in the performance capabilities, reducing complications, and improving subject/group/company adherence to the work program and slow aging processes.

According to some embodiments, device-generated maneuvers/stimulation techniques include mechanical, magnetic, electric, electromagnetic, ultrasound, thermal, or the like, which can improve performance and prevent burnout. According to some embodiments, changes in the tasks and regimens include a change in any parameter of the regimens, including length of working session, degree of difficulty, change in order of independent tasks, and similar parameters relevant to performance. The device-generated maneuvers/stimulation characteristics include variations or changes in regimens and maneuver/stimulation patterns (repetitions), frequency, intensity, duration, or any other parameter controlled for these. According to some embodiments, the regimens and device-generated maneuvers may be provided continuously or intermittently with On/Off periods, and the duration of the periods and/or the ratio between them may be changed in either a structured manner, randomly or semi-randomly.

According to some embodiments, the device is configured to be placed at a desired position on the participant's body to induce maneuver/stimulation.

According to some embodiments, challenged regimens and maneuver/stimulation devices communicate with an update module, such as a learning machine, for continuously updating regimens and/or maneuver parameters/characteristics. According to some embodiments, the communication may be wireless.

According to some embodiments, both external and internal devices can be used for data collection and input of data from various sources and/or for the continuous generation of the new regimen and the new device-generated maneuver/stimuli required for achieving a target goal and for continuously improving the overall performance and for prevention and/or slowing of aging processes. The closed loop system continuously or semi-continuously receives data from internal and external measured parameters from one or many users. It is continuously being processed by the controller for generating a new regimen or a new maneuver/stimuli to be administered to the user via an internal or external device. Optional sensors convey data to the processor that both conveys and is fed data by a cell phone, a cloud, and possibly a computer and/or a stimulator device. According to some embodiments, the update-unit/learning machine is updated upon changes in the measured information, for example, if the change is more significant than a certain percentage of the previous value if the values reach a predetermined threshold or any combination of the above.

Disclosed herein is an example of using a closed-loop continuous learning algorithm to prevent adaptation for task performance and slowing aging.

The target treatment is improved performance of any task capability within a pre-defined period and/or with a lower effort.

The system can enable reaching a target with less effort while improving adherence, overcoming burnout, and preventing or slowing down aging processes.

The regimen algorithm and/or stimulation device (internal or external device) receives data from the sensors (internal and or external), indicative of the overall performance of the subject, pulse, breathing, oxygen saturation, blood pressure, skin conductivity along with additional tests and parameters which are relevant or irrelevant to activity, to the task, and to function, or to aging.

The input data is processed in correlation with the target of the function to assess whether an improvement was achieved and to what extent following each period. If no improvement toward the target is achieved, a new regimen and/or device-generated maneuver/stimuli is being generated. Suppose a positive step towards the target is achieved. In that case, the controller will divide each type of regimen (including the degree of difficulty, speed, and additional parameters that are controlled by the algorithm) and/or the selected maneuver/stimuli (electrical, mechanical, magnetic, ultrasound) into 100 percentiles and determines the percentile for each of the components of the regimen (such as time and degree of difficulty of the task within a predetermined range) and/or maneuver/stimuli (such as rate of stimuli, rhythm, power, frequency, amplitude, and temperature or others or any combination thereof) and which order of administration or alternating between them was the most efficient in contributing to the achievement of the target change that improved the performance. Based on that analysis, a continuous new regimen and/or device-generated maneuver/stimuli is generated. The machine learning computer-implemented method may require several samples for learning the user and providing effective stimulations.

The output regimen and/or device-generated maneuvers or stimulation parameters update mechanism/algorithm is configured to continuously narrow or widen the range or change the order by which the regimen is administered to be targeted on the most effective regimen and/or maneuver/stimulation characteristics for the specific user/group of users/company. Narrowing or widening the range for each parameter will keep the randomization within a predefined, continuously changing range.

The output-challenged-regimen and/or the output device-generated maneuvers or stimulation characteristics/parameters update mechanism/algorithm is configured to learn from indications/measurements (measured parameters), which may or may not be directly related to the targets. These include, for example, any relevant or irrelevant parameters to said function.

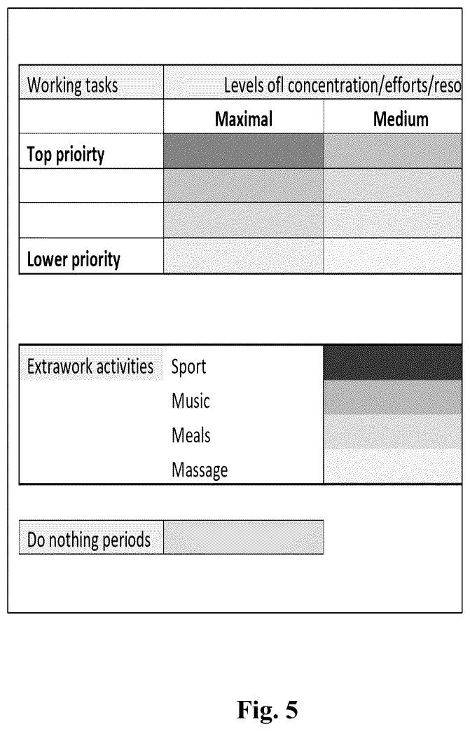

Reference is now made to FIG. 5, which schematically illustrates three task table types. It includes work task tables sorted by the concentration levels/efforts/resources required for execution. Each type of task is prioritized, and the amount of time required for its accomplishment is specified. The second type is tables for extra work activities, including sports, music, meals, massage, etc. The third type is tables, which include “do nothing periods” as part of the daily schedule or in which no task is allocated.

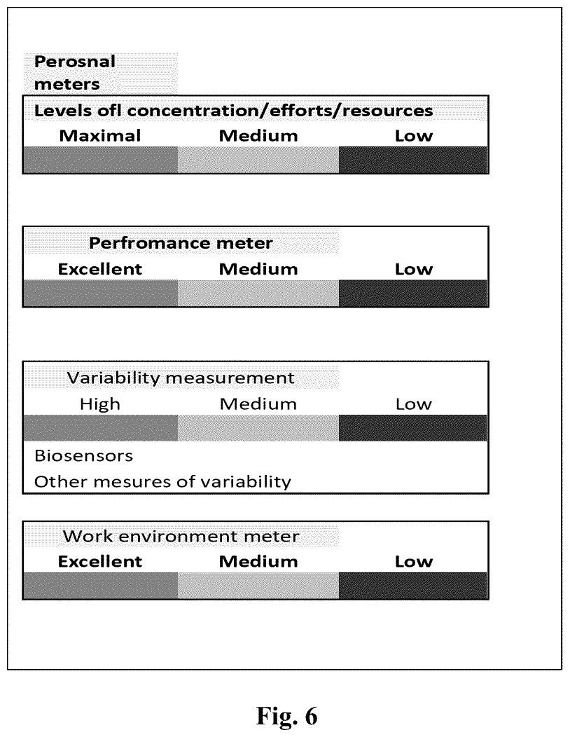

Reference is now made to FIG. 6, which schematically illustrates four types of meters: A personal meter where the individual, the team and/or the company specify their concentration level, efforts, energy, and resources they anticipate for each period during the day. The second is a performance meter that measures the efficiency and overall goal achievement. The third is a variability meter that measures the degree of variability in an individual, team, or company. The fourth is a work environment meter based on inputs from every team member and the management.

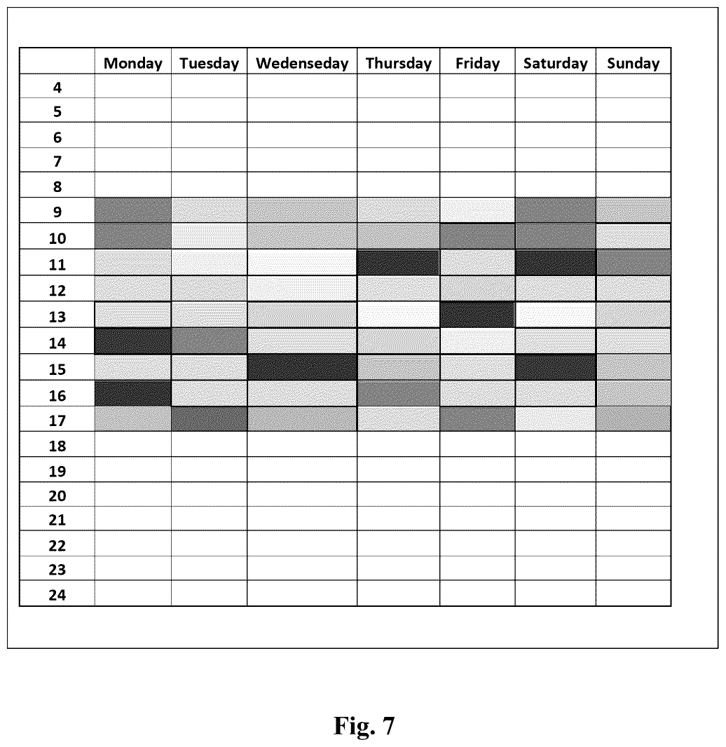

Reference is now made to FIG. 7, which schematically illustrates a calendar based on the algorithm using the task tables and the meters. The algorithm prepared an example of a calendar using the color code shown in FIGS. 5 and 6. In this example, the algorithm inserts the tasks based on the tables and matches them together based on the different levels of the algorithm used.

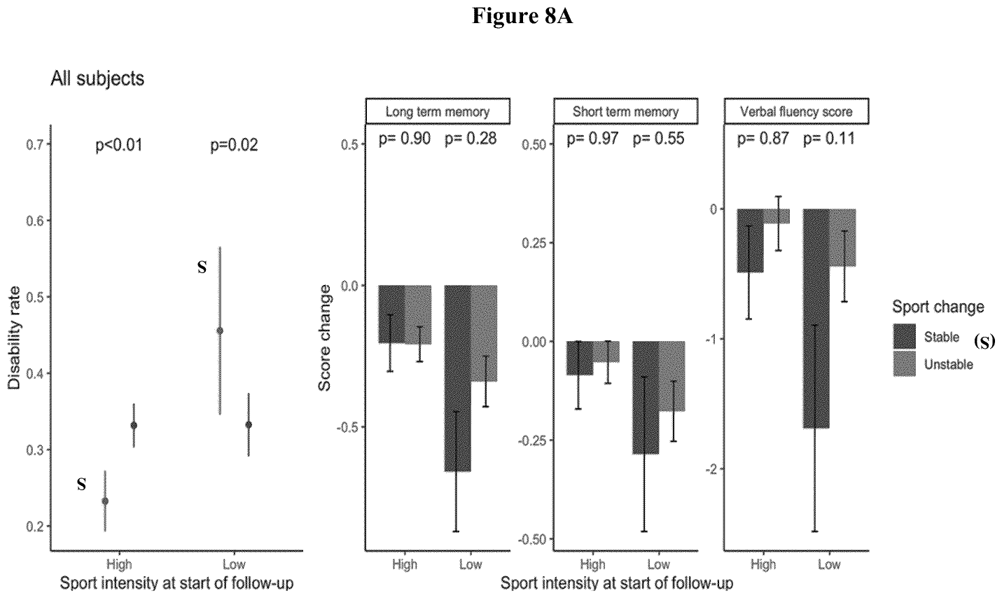

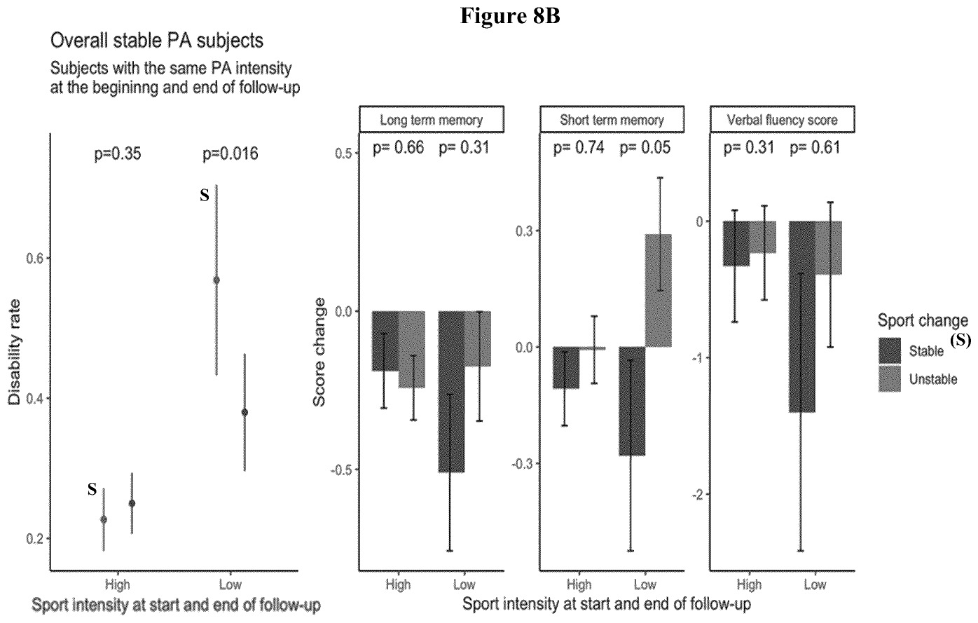

Reference is now made to FIGS. 8A-B, which schematically illustrates a subject/group of subjects/company-tailored, continuously developing, randomization-based algorithm for preventing or slowing aging processes. Introducing. Regular physical activity (PA) promotes mental and physical health. The association between intra-individual variability in PA and disability among non-athlete adults was documented. The figure shows the association between intra-individual variability in PA frequency with physical and cognitive disability: FIG. 8A shows the disability rates at the end of follow-up (left) and change in cognitive function by the end of follow-up (right) among the entire study cohort, across high and low sport intensity at baseline. FIG. 8B Shows the same analysis as in FIG. 8A, among the subset of and end of follow-up.

Non-disabled adults aged>50 followed over 14 years. Self-reported PA frequency was documented bi- to triennially. Low PA intensity was defined as vigorous PA frequency less than once a week. Stable(S) PA was defined as an unchanged PA intensity in all consecutive middle observations. The primary outcome was defined as a physical limitation in everyday activities at the end of the survey. Secondary outcomes were cognitive functions, including short-term memory, long-term memory, and verbal fluency. The study included 2,049 non-disabled adults with a mean age of 53 and 49.1% women in the initially low PA intensity group; variability was associated with a reduced physical disability (45.6% vs. 33.3%, stable vs. unstable PA; P=0.02; adjusted P=0.03). Among individuals with the same low PA intensity at the beginning and end of follow-up, variability was associated with reduced physical disability (56.9% vs. 36.5%, stable vs. unstable PA; P=0.02; adjusted P=0.04) and improved short-term memory (score change: −0.28 vs. +0.29, stable vs. unstable PA; P=0.05). The data shows that incorporating variability into PA regimens of inactive adults may enhance their physical and cognitive benefits.

According to some embodiments, the algorithm operated in the update module may consider outliers from the plurality of users, to which general users may not fit, and develop new models of regimens (new decision structures) for such outliers.

The algorithm, per one subject, and/or a group of subjects, and/or a company or several companies may be developed based on big data analysis generated from multiple sources. It is noted that the new regimen and/or the new maneuver/stimuli regimen generated by the big data can be further analyzed by type of function, by subject performance, by associated task, by background and environmental conditions such as the environment in work, and subject/team/company related factors such as previous scores, tests relevant to the target function, age, gender, body weight, a delta of change in the target parameter (e.g. performance capability of a said task) over time, geographic location, and other target and/or subject and/or type of parameters, it may not be identical per all subjects/groups/companies, and not identical for the same subject/group/company under changing conditions. It is only a contributing level of data to the deep machine learning algorithm, which generates a subject and/or a team and/or a company-specific algorithm.

The output regimen can be based on an open or a closed loop system in which a plurality of features is received initially, and machine learning algorithms are applied. Output parameters are then determined and added as additional feature plurality or used to update the output parameters, which are then added as additional feature plurality.

According to some embodiments, the algorithm may change over time per each subject/team/company, such that an improvement in the capability to a certain degree may not require the same regimen and/or maneuver/stimuli needed for achieving the previous level. As the algorithm continuously or semi-continuously learns, it continuously changes based on the data accumulated by the big data and from each subject and other subjects.

For example, suppose a subject suffers from burnout or from a “doing more of the same syndrome,” wishes to improve their capability, and/or lost the effect of extra work activities, wants to slow or prevent aging processes, and/or is not improving with a regular-perpetual gradual regimen. In that case, they can use one of the following or any combination of the following to improve their performance, prevent loss of the effect of the regular regimens, or maximize the ongoing effect of the regimen:
a. Use a subject/group/company-specific algorithm that determines the irregularity of all parameters of the challenged regimens relevant to the target function and task by inducing a deep machine learning closed loop algorithm-based irregularity associated with the said regimen.
b. Use a maneuver/stimulation-generating device that can be put on any other organ that delivers any mechanical, electrical, ultrasound-based, temperature-based, or any other type of stimuli in addition to the chronic work regimen.
c. Use of an algorithm of any combination of the above.

The terminology used herein only describes particular embodiments and is not intended to be limiting. As used herein, the singular forms “a,” “an,” and “the” are intended to include the plural forms as well unless the context indicates otherwise. It is further understood that the terms “comprises” or “comprising,” when used in this specification, specify the presence of stated features, integers, steps, operations, elements, or components but do not preclude or rule out the presence or addition of one or more other features, integers, steps, operations, elements, components, or groups thereof.

While several exemplary aspects and embodiments have been discussed above, those of skill in the art will recognize certain modifications, additions, and sub-combinations. Therefore, it is intended that the following appended claims and claims hereafter introduced be interpreted to include all such modifications, additions, and sub-combinations within their true spirit and scope.

What we claim is:

1. A method for improving function and performance and/or for preventing, mitigating, or overcoming partial or complete loss of effect of regimen, due to adaptation to the regimen; and/or partial or complete loss of effect of device-generated maneuvers or stimulations administered to or used by a subject in need thereof, or non-responsiveness to challenged-regimens, and/or maximizing the effect of regimens or maneuvers, the method comprising:
receiving a plurality of physiological or pathological parameters of the subject and/or information from the subject, a team and/or a company, and/or a device;
applying an open or a closed loop machine learning algorithm on the plurality of physiological or pathological parameters;
determining output parameters relating to subject, team and/or company-specific challenged regimens, for facilitating a continuous improvement of the regimen or device-based maneuver or stimulation, wherein the output parameters comprise regimen or maneuver parameters, thereof,
utilizing a subject, team and/or company-tailored, continuously or semi-continuously randomization-based or non-randomization-based algorithm, for continually improving performance; and
utilizing a subject-tailored, continuously or semi-continuously developing a randomization-based or non-randomization-based algorithm capable of mixing two or more work tasks, whether relevant to the target for improving function.

2. The method of claim 1, further comprising updating output parameters comprising, challenged-regimens-related parameters; and/or device-generated maneuver or stimulation parameters comprising amplitude, frequency, interval, and/or duration; or any combinations thereof, comprising updating changes and alterations in each of the parameters, which are of relevance to target performance.

3. The method of claim 1, further comprising determining challenged regimens and/or maneuvers or stimulation parameters.

4. The method of claim 1, further comprising updating regimen parameters based on data being continuously or semi-continuously learned from user(s).

5. The method of claim 1, wherein the machine learning algorithm further considers personal and/or group and/or company data selected from sources comprising subject, team and/or company performance, function-related scores, parameters relevant to performance, age, weight, tasks, gender, ethnicity, geography, pathological history and/or state, temperature, metabolic rate, brain function, health status, heart, lung muscle function, blood tests, and physiological or pathological biomarkers or parameters that can be measured, directly or indirectly associated with the physiological target or the task and/or the subject, team and/or company.

6. The method of claim 1, wherein at least one of the physiological or pathological parameters is obtained from a sensor.

7. The method of claim 1, wherein the subject/team/company challenged-regimen, is based on a deep machine learning closed loop-irregularity, regularity, randomization, or non-randomization.

8. The method of claim 1, comprising notifying the subject/team/company in real-time.

9. The method of claim 1, further comprising challenged-regimens and/or maneuvers or stimulating-generating devices to evoke a reaction by a form of external, wearable, swallowed and/or implanted device associated with improving function.

10. The method of claim 1 further comprising administering the challenged regimen to the subject/team/company.

11. The method of claim 1, for improving function in healthy subjects who wish to improve performance, and/or for reaching a better target, for prevention or slowing down of aging processes, and/or for improving the effect of anti-aging drugs, maneuvers and/or techniques.

12. The method of claim 1, where challenged regimens, are utilized in combination with device-generated maneuvers or stimulation parameters, or with regimens of conditions wherein enhanced functioning is required, or for improving performance, for prevention or overcoming of adaptation to chronic regimens, or for continuously overcoming partial/complete loss of an effect of these regimens, and/or for improving the beneficial effects of a regimen.

13. A system for preventing, mitigating and/or treating partial/complete loss of effect due to adaptation to a challenged-regimen and/or used in combination with device-generated maneuvers or stimulation parameters, administered to or used by a subject, team and/or a company in need thereof, or non-responsiveness to regimens, and continuously maximizing the beneficial effect of work regimens, and/or improving function, the system being continuous, semi-continuous, conditional or non-continuous closed loop, comprising one or more processing units configured for
receiving, a plurality of physiological or pathological parameters of the subject, team and/or company, and/or information therefrom and/or device, or other sources;
applying a closed-loop machine learning algorithm on the plurality of physiological or pathological parameters;
determining output parameters relating to subject, team. and/or company-specific challenged-regimens, and/or in combination with device-generated maneuvers/stimulation parameters, for facilitating improvement of work regimens or device-based maneuvers, wherein the output parameters comprise regimen administration parameters, maneuver/stimulation parameters, or any combination thereof;
using a subject/group of subjects/company-tailored continuously, semi-continuously, and non-continuous information for developing randomization-based or non-randomization-based algorithms for improving function following challenged regimens and/or any maneuver that can improve the function for continuously improving the performance related to the function of the said subject/group/company;
using a subject/team/company-tailored continuously or semi-continuously developing a randomization-based algorithm that mixes two or more tasks, whether relevant to the task.

14. The system of claim 13, wherein the machine learning algorithm is further configured to update output parameters comprising challenged-regimen and/or administration, and precisely parameters which are relevant to the regimens which are specific for the task and/or to stimulation signals; based on initial regimens parameters and/or initial stimulation parameters and/or on continuous or semi-continuous information obtained during or following the challenged-working session, and/or a maneuver which can improve the function by overcoming adaptation to maneuvers or regimes.

15. The system of claim 13, wherein the machine learning algorithm further considers subject/team/company data selected from the data comprising subject/team/company performance, task-related scores, parameters relevant to performance, and physiological or pathological biomarkers or parameters that can be measured, directly or indirectly associated with the physiological target or with function and/or health status of the subject and/or subject's chronic condition that can be measured, directly or indirectly associated with the target to be achieved continuously.

16. The system of claim 13, wherein at least one of the physiological or pathological parameters is obtained from a sensor.

17. The system of claim 13, wherein the subject regimen or any type of maneuver/regimen/regimens is irregular.

18. The system according to claim 13, wherein processor configured to notify the subject/team/company regarding regimen and/or device-generated maneuvers/stimulation regimens-relevant parameters, including relevant and irrelevant work-related parameters for administering these regimens.

19. The system of claim 13, further comprising a processor configured to use a work regimen and/or to improve function or manipulate/stimulate an organ of the subject/group to evoke a reaction by a form of external, wearable, swallowed and/or an implanted device.

20. The system of claim 13, wherein a closed algorithm receives input from a subject, groups of subjects, or companies, for determining a change of challenged regimen relevant to improving the target or non-target function by said regimens.
